SEQUENCE LISTING (65 sequences)

SEQ ID NO: 1 - protein, 318 aa, Artificial sequence
  Recombinant Hyr1 protein amino acid sequence
METDTLLLWVLLLWVPGSTGGSGHHHHHHGEVEKGASLFIKSDNGPVLALNVALSTLVRPVINNGVISLNSKSSTSFSNFDIGGSSFTNNGEIYLASSGLVKSTAYLYAREWTNNGLIVAYQNQKAAGNIAFGTAYQTITNNGQICLRHQDFVPATKIKGTGCVTADEDTWIKLGNTILSVEPTHNFYLKDSKSSLIVHAVSSNQTFTVHGFGNGNKLGLTLPLTGNRDHFRFEYYPDTGILQLRAAALPQYFKIGKGYDSKLFRIVNSRGLKNAVTYDGPVPNNEIPAVCLIPCTNGPSAPESESDLNTPTTSSIGT

SEQ ID NO: 2 - protein, 122 aa, Homo sapiens
EVQLVQSGGGLVQPGGSLGLSCAASGFIFTSYAMTWVRQAPGKGLEWVSVITGNVGTSYYADSVKGRFTISRDNSKKTVSLQMNSLRAEDTAIYYCVKTRYDFSSGYYFDDWGQGTLVSVSS

SEQ ID NO: 3 - protein, 123 aa, Homo sapiens
EVQLVESGGTLVQPGGSLRLSCAASGFTFSDYWMNWVRQAPGKGLEWVANIKQDGSEKYYVDSLRGRVTISRDNAQNSVFLQMHSLSVEDTAVYYCARDGYTFGPATTELDHWGRGTLVSVSS

SEQ ID NO: 4 - protein, 121 aa, Homo sapiens
EVQLVQSGGGLAQPGRSLRLSCAASGFGFDDFAMHWVRQPPGKGLEWVSGLTWNGGSIDYAGSVRGRFTISRDNAKNSLFLQMNSLRAEDTALYYCAKGLSGGTMAPFDIWGQGTMVSVSS

SEQ ID NO: 5 - protein, 118 aa, Homo sapiens
EVQLLESGGGVVQPGRSLRLSCAASGFTFSNYGMHWVRQAPGKGLEWVAVVWFDGSYKYYTDSVKGRFTISRDNSKSTLYLQMNSLRAEDTAVYYCVSPIMTSAFDIWGPGTMVSVSS

SEQ ID NO: 6 - protein, 107 aa, Homo sapiens
DIVMTQSPSSVSASVGDKVTITCRASQGISRWLAWYQQKPGEAPELLIYAASSLQSGVPSRFSGSGSGTDFTLTISSLQPEDFATYYCQQANSFPITFGQGTRLQIK

SEQ ID NO: 7 - protein, 106 aa, Homo sapiens
QLVLTQPPSVSVSPGQTASITCSGDELRNKYTSWYQQKSGQSPVLVIYQDNNRPSGIPERFSGSQSGDTATLTISGTQAVDEADYYCQAWVSQTLVFGGGTKLTVL

SEQ ID NO: 8 - protein, 109 aa, Homo sapiens
QAGLTQPPSVSVAPGQTATIPCGGNNIGSKHVHWYQQKPGQAPVAVVYDDSDRPSGVPERFSGSNSGNTATLTISSVEAGDEADYYCQVWDRSSDHFWLFGGGTRLTVL

SEQ ID NO: 9 - protein, 109 aa, Homo sapiens
QLVLTQPPSASGSPGQSVTISCTGTSSDVGGSNFVSWYQHHPGKAPKLMIYEVSQRPSGVPDRFSGSKSGNTASLTVSGLQADDEADYYCSSYAGSVVLFGGGTKLTVL

SEQ ID NO: 10 - protein, 29 aa, Homo sapiens
EVQLVQSGGGLVQPGGSLGLSCAASGFIF

SEQ ID NO: 11 - protein, 6 aa, Homo sapiens
TSYAMT

SEQ ID NO: 12 - protein, 14 aa, Homo sapiens
WVRQAPGKGLEWVS

SEQ ID NO: 13 - protein, 17 aa, Homo sapiens
VITGNVGTSYYADSVKG

SEQ ID NO: 14 - protein, 32 aa, Homo sapiens
RFTISRDNSKKTVSLQMNSLRAEDTAIYYCVK

SEQ ID NO: 15 - protein, 13 aa, Homo sapiens
TRYDFSSGYYFDD

SEQ ID NO: 16 - protein, 11 aa, Homo sapiens
WGQGTLVSVSS

SEQ ID NO: 17 - protein, 23 aa, Homo sapiens
DIVMTQSPSSVSASVGDKVTITC

SEQ ID NO: 18 - protein, 11 aa, Homo sapiens
RASQGISRWLA

SEQ ID NO: 19 - protein, 15 aa, Homo sapiens
WYQQKPGEAPELLIY

SEQ ID NO: 20 - protein, 7 aa, Homo sapiens
AASSLQS

SEQ ID NO: 21 - protein, 32 aa, Homo sapiens
GVPSRFSGSGSGTDFTLTISSLQPEDFATYYC

SEQ ID NO: 22 - protein, 9 aa, Homo sapiens
QQANSFPIT

SEQ ID NO: 23 - protein, 10 aa, Homo sapiens
FGQGTRLQIK

SEQ ID NO: 24 - protein, 29 aa, Homo sapiens
EVQLVESGGTLVQPGGSLRLSCAASGFTF

SEQ ID NO: 25 - protein, 6 aa, Homo sapiens
SDYWMN

SEQ ID NO: 26 - protein, 14 aa, Homo sapiens
WVRQAPGKGLEWVA

SEQ ID NO: 27 - protein, 17 aa, Homo sapiens
NIKQDGSEKYYVDSLRG

SEQ ID NO: 28 - protein, 32 aa, Homo sapiens
RVTISRDNAQNSVFLQMHSLSVEDTAVYYCAR

SEQ ID NO: 29 - protein, 14 aa, Homo sapiens
DGYTFGPATTELDH

SEQ ID NO: 30 - protein, 11 aa, Homo sapiens
WGRGTLVSVSS

SEQ ID NO: 31 - protein, 22 aa, Homo sapiens
QLVLTQPPSVSVSPGQTASITC

SEQ ID NO: 32 - protein, 11 aa, Homo sapiens
SGDELRNKYTS

SEQ ID NO: 33 - protein, 15 aa, Homo sapiens
WYQQKSGQSPVLVIY

SEQ ID NO: 34 - protein, 7 aa, Homo sapiens
QDNNRPS

SEQ ID NO: 35 - protein, 32 aa, Homo sapiens
GIPERFSGSQSGDTATLTISGTQAVDEADYYC

SEQ ID NO: 36 - protein, 9 aa, Homo sapiens
QAWVSQTLV

SEQ ID NO: 37 - protein, 10 aa, Homo sapiens
FGGGTKLTVL

SEQ ID NO: 38 - protein, 29 aa, Homo sapiens
EVQLVQSGGGLAQPGRSLRLSCAASGFGF

SEQ ID NO: 39 - protein, 6 aa, Homo sapiens
DDFAMH

SEQ ID NO: 40 - protein, 14 aa, Homo sapiens
WVRQPPGKGLEWVS

SEQ ID NO: 41 - protein, 17 aa, Homo sapiens
GLTWNGGSIDYAGSVRG

SEQ ID NO: 42 - protein, 32 aa, Homo sapiens
RFTISRDNAKNSLFLQMNSLRAEDTALYYCAK

SEQ ID NO: 43 - protein, 12 aa, Homo sapiens
GLSGGTMAPFDI

SEQ ID NO: 44 - protein, 11 aa, Homo sapiens
WGQGTMVSVSS

SEQ ID NO: 45 - protein, 22 aa, Homo sapiens
QAGLTQPPSVSVAPGQTATIPC

SEQ ID NO: 46 - protein, 11 aa, Homo sapiens
GGNNIGSKHVH

SEQ ID NO: 47 - protein, 15 aa, Homo sapiens
WYQQKPGQAPVAVVY

SEQ ID NO: 48 - protein, 7 aa, Homo sapiens
DDSDRPS

SEQ ID NO: 49 - protein, 32 aa, Homo sapiens
GVPERFSGSNSGNTATLTISSVEAGDEADYYC

SEQ ID NO: 50 - protein, 12 aa, Homo sapiens
QVWDRSSDHFWL

SEQ ID NO: 51 - protein, 10 aa, Homo sapiens
FGGGTRLTVL

SEQ ID NO: 52 - protein, 29 aa, Homo sapiens
EVQLLESGGGVVQPGRSLRLSCAASGFTF

SEQ ID NO: 53 - protein, 6 aa, Homo sapiens
SNYGMH

SEQ ID NO: 54 - protein, 14 aa, Homo sapiens
WVRQAPGKGLEWVA

SEQ ID NO: 55 - protein, 17 aa, Homo sapiens
VVWFDGSYKYYTDSVKG

SEQ ID NO: 56 - protein, 32 aa, Homo sapiens
RFTISRDNSKSTLYLQMNSLRAEDTAVYYCVS

SEQ ID NO: 57 - protein, 9 aa, Homo sapiens
PIMTSAFDI

SEQ ID NO: 58 - protein, 11 aa, Homo sapiens
WGPGTMVSVSS

SEQ ID NO: 59 - protein, 22 aa, Homo sapiens
QLVLTQPPSASGSPGQSVTISC

SEQ ID NO: 60 - protein, 14 aa, Homo sapiens
TGTSSDVGGSNFVS

SEQ ID NO: 61 - protein, 15 aa, Homo sapiens
WYQHHPGKAPKLMIY

SEQ ID NO: 62 - protein, 7 aa, Homo sapiens
EVSQRPS

SEQ ID NO: 63 - protein, 32 aa, Homo sapiens
GVPDRFSGSKSGNTASLTVSGLQADDEADYYC

SEQ ID NO: 64 - protein, 9 aa, Homo sapiens
SSYAGSVVL

SEQ ID NO: 65 - protein, 10 aa, Homo sapiens
FGGGTKLTVL

(19) United States
(12) Patent Application Publication
(10) Pub. No.: US 2022/0153818 A1
(43) Pub. Date: May 19, 2022

(54) ANTIBODY MOLECULES AND USES THEREOF

(71) Applicant: THE UNIVERSITY COURT OF THE UNIVERSITY OF ABERDEEN, Aberdeenshire (GB)

(72) Inventors: Fiona Marion RUDKIN, Aberdeenshire (GB); Neil Andrew Robert GOW, Aberdeenshire (GB); Louise Ann WALKER, Aberdeenshire (GB)

(21) Appl. No.: 17/435,615

(22) PCT Filed: Feb. 27, 2020
(86) PCT No.: PCT/EP2020/055134

§ 371 Date: Sep. 1, 2021

(30) Foreign Application Priority Data
Mar. 1, 2019 (GB) 1902769.7

(51) Int. Cl. C07K 16/12; C07K 16/14; A61K 47/68; A61K 45/06; A61K 39/395; A61P 31/04; G01N 33/569; G01N 33/543

(52) U.S. Cl. CPC C07K 16/1217; C07K 16/14; A61K 47/6835; A61K 45/06; A61K 39/39575; A61P 31/04; C07K 2317/622; G01N 33/54388; C07K 2317/14; C07K 2317/21; C07K 2317/565; C07K 2317/92; G01N 33/56911

(57) ABSTRACT

This invention relates to recombinant human antibody molecules for use in a method of treatment of Acinetobacter infection. The antibodies bind Acinetobacter antigens, for example from Acinetobacter spp. Human antibody encoding genes targeting clinically relevant Candida epitopes have been isolated from single B cells from carefully selected donors and screened with specified types of protein or cell wall extract. The panel of purified, fully human recombinant IgG1 mAbs generated displayed a diverse range of specific binding profiles and demonstrated efficacy in a disease model. The fully human mAbs and derivatives thereof have utility in the generation of diagnostics, therapeutics and vaccines.

### TECHNICAL FIELD

This invention relates to recombinant human antibody molecules. The antibodies bind Acinetobacter antigens, for example A. baumannii spp. Such antibody molecules find use in the treatment, diagnosis and/or detection of Acinetobacter infections.

### BACKGROUND ART

A. baumannii is one of the most common and antibiotic-resistant pathogens encountered throughout the world. Infections are most prevalent in immunocompromised patients in the intensive care unit setting and include pneumonia, urinary tract infections and bloodstream infections. Acinetobacter is also a major cause of combat-associated wound infections (Perez F et al. Global challenge of multidrug-resistant Acinetobacter baumannii. Antimicrob Agents Chemother 2007). Approximately 50%-70% of clinical isolates are extensively drug resistant (XDR) resulting in >10 000 deaths per year in the US alone (Nielsen et al. Monoclonal antibody protects against Acinetobacter baumannii infection by enhancing bacterial clearance and evading sepsis. J Infect Dis 2017). There are only a limited number of new drugs in development.

Travis et al (2017) describe a monoclonal antibody based therapy that is highly protective during bloodstream and lung infections, by defining it's in vitro and in vivo antibacterial effects, including in combination with a standard anti-A. baumannii antibiotic. The monoclonal antibody was raised by immunizing mice with sublethal inocula of a hypervirulent bacterial clinical isolate and was shown to enhance opsonophagocytosis (Travis et. al “Monoclonal Antibody Protects Against Acinetobacter baumannii Infection by Enhancing Bacterial Clearance and Evading Sepsis”, The Journal of Infectious Diseases, Volume 216, Issue 4, 15 Aug. 2017, Pages 489-501).

Nevertheless it can be seen that novel sources of diagnostic and therapeutic reagents targeting Acinetobacter pathogens would provide a contribution to the art.

### DISCLOSURE OF THE INVENTION

The present invention seeks to provide novel diagnostics and therapeutics for Acinetobacter infections, through a mAb-based approach using antibodies raised against C. albicans cell wall proteins.

The inventors previously isolated human antibody encoding genes targeting clinically relevant Candida epitopes from single B cells that were derived from donors with a history of mucosal Candida infections and screened with recombinant Candida albicans Hyr1 cell wall protein, as disclosed in WO2016/142600, which is hereby incorporated by reference. The panel of purified, fully human recombinant IgG1 mAbs generated demonstrated efficacy in a murine model of disseminated candidiasis.

Surprisingly, it has now been found that some of these mAbs opsonise bacterial A. baumannii cells for phagocytosis by immune cells. The panel of purified, fully human recombinant IgG1 mAbs generated display a diverse range of specific binding profiles to pathogenic bacteria and demonstrate efficacy in a Galleria mellonella model of disseminated A. baumannii infection. The fully human mAbs have utility in the generation of diagnostics, therapeutics and vaccines.

WO 2013/04078 previously disclosed that fragments of the fungal Candida cell surface protein Hyr1 are useful in immunising a subject against Candida infections. It showed that pooled IgG raised against 8 Hyr1 peptides directly neutralise function of Hyr1p in resisting phagocyte killing of such fungal cells rather than enhancing opsonophagocytosis. The same document also proposed that such antibodies might prevent infections by Acinetobacter baumannii. It had also been proposed that Hyr1 shares homology with certain cell surface proteins of Acinetobacter baumannii including hemagglutin (FhaB) and outer membrane protein A (OmpA), and that antibodies raised against peptide motifs of Hyr1 mitigates mixed A. baumannii/C. albicans biofilms formation (Uppuluri et al., The Hyr1 protein from the fungus Candida albicans is a cross kingdom immunotherapeutic target for Acinetobacter bacterial infection. PLOS pathogens 2018).

Despite these general disclosures, it could not have been predicted that certain anti-Hyr1 antibodies were capable of providing the advantageous combination of therapeutic properties described for the antibodies of the present invention.

Thus a first aspect of the invention provides an isolated recombinant human anti-Candida antibody molecule derived from single B cells for use in a method of treatment of an Acinetobacter bacterial infection, wherein the antibody molecule comprises a VH domain comprising a HCDR3 having the amino acid sequence of SEQ ID NO: 6x or the sequence of SEQ ID NO: 6x with 1, 2, or 3 amino acid substitutions, deletions or insertions,
wherein ‘x’ is one letter from F, E, D and C, and said sequence is as shown in Table ‘x’ herein.

In some embodiments, the antibody molecule for use comprises an HCDR2 having the amino acid sequence of SEQ ID NO: 4x or the sequence of SEQ ID NO: 4x with 1, 2, or 3 amino acid substitutions, deletions or insertions.

In some embodiments, the antibody molecule comprises an HCDR1 having the amino acid sequence of SEQ ID NO: 2x or the sequence of SEQ ID NO: 2x with 1, 2 or 3 amino acid substitutions, deletions or insertions.

In some embodiments, the antibody molecule comprises a VH domain comprising a HCDR1, a HCDR2 and a HCDR3 having the sequences of SEQ ID NOs 2x, 4x and 6x respectively.

In some embodiments, the antibody molecule comprises a VH domain comprising one or more or all of a FW1, a FW2, a FW3 and a FW4 having the sequences of SEQ ID NOs lx, 3x, 5x and 7x respectively.

In some embodiments, the antibody molecule comprises a VH domain having an amino acid sequence at least about 80% identical to SEQ ID NO: 15x and\or having the amino acid sequence of SEQ ID NO: 15x and\or the sequence of SEQ ID NO: 15x with 1, 2, 3, 4, 5, 6, 7, 8, 9, or 10 amino acid substitutions, deletions or insertions in SEQ ID NO: 15x.

In some embodiments, the antibody molecule comprises a VL domain comprising LCDR1, LCDR2 and LCDR3 having the sequences of SEQ ID NOs 9x, 11x and 13x respectively, or the sequences of SEQ ID NOs 9x, 11x and 13x respectively with, independently, 1, 2 or 3 or more amino acid substitutions, deletions or insertions.

In some embodiments, the antibody molecule comprises a VL domain comprising LCDR1, LCDR2 and LCDR3 having the sequences of SEQ ID NOs 9x, 11x and 13x respectively.

In some embodiments, the antibody molecule comprises a VL domain comprising one or more or all of a FW1, a FW2, a FW3 and a FW4 having the sequences of SEQ ID NOs 8x, 10x, 12x and 14x respectively.

In some embodiments, the antibody molecule comprises a VL domain having an amino acid sequence at least about 80% identical to SEQ ID NO: 16x and\or having the sequence of SEQ ID NO: 16x and\or the sequence of SEQ ID NO: 16x with 1 or more, for example 2, 3, 4, 5, 6, 7, 8, 9, 10 or more amino acid substitutions, deletions or insertions in SEQ ID NO: 16x.

In some embodiments, the antibody molecule comprises a VH domain comprising a HCDR1, a HCDR2 and a HCDR3 having the sequences of SEQ ID NOs 2x, 4x, and 6x, respectively, and a VL domain comprising a LCDR1, a LCDR2 and a LCDR3 having the sequences of SEQ ID NOs 9x, 11x and 13x, respectively.

In some embodiments, antibody molecule comprises VH and VL domains having the amino acid sequences of SEQ ID NO: 15x and SEQ ID NO: 16x respectively.

In some embodiments, the Acinetobacter bacterial infection is an A. baumannii infection.

In some embodiments, the Acinetobacter bacterial infection is an A. pittii infection, an A. nosocomialis infection, an A. calcoaceticus infection, an A. seifertii infection or an A. dijkshoorniae infection.

In some embodiments, the antibody molecule binds A. baumannii with an EC₅₀ value of 1 to 1500 ng/mL. In some embodiments, the antibody molecule binds A. baumanii with an EC₅₀ value of less than 1500 ng/mL, less than 1400 ng/mL, less than 1300 ng/mL, less than 1200 ng/mL, less than 1100 ng/mL, less than 1000 ng/mL, less than 900 ng/mL, less than 800 ng/mL, less than 700 ng/mL, less than 600 ng/mL, less than 500 ng/mL, less than 400 ng/mL, less than 300 ng/mL, less than 200 ng/mL, less than 100 ng/mL, less than 90 ng/mL, less than 80 ng/mL, less than 70 ng/mL, less than 60 ng/mL, less than 50 ng/mL, less than 40 ng/mL, less than 30 ng/mL, less than 20 ng/mL, less than 10 ng/mL, less than 5 ng/mL, less than 4 ng/mL, less than 3 ng/mL, less than 2 ng/mL or less than 1 ng/mL.

In some embodiments, the antibody molecule binds A. pittii, A. nosocomialis, A. calcoaceticus, A. seifertii or A. dijkshoorniae with an EC₅₀ value of 1 to 1500 ng/mL. In some embodiments, the antibody molecule binds A. pittii, A. nosocomialis, A. calcoaceticus, A. seifertii or A. dijkshoorniae with an EC₅₀ value of less than 1500 ng/mL, less than 1400 ng/mL, less than 1300 ng/mL, less than 1200 ng/mL, less than 1100 ng/mL, less than 1000 ng/mL, less than 900 ng/mL, less than 800 ng/mL, less than 700 ng/mL, less than 600 ng/mL, less than 500 ng/mL, less than 400 ng/mL, less than 300 ng/mL, less than 200 ng/mL, less than 100 ng/mL, less than 90 ng/mL, less than 80 ng/mL, less than 70 ng/mL, less than 60 ng/mL, less than 50 ng/mL, less than 40 ng/mL, less than 30 ng/mL, less than 20 ng/mL, less than 10 ng/mL, less than 5 ng/mL, less than 4 ng/mL, less than 3 ng/mL, less than 2 ng/mL or less than 1 ng/mL.

In some embodiments, the antibody molecule binds A. baumanii with an EC₅₀ value of less than 20 ng/mL.

In some embodiments, the antibody molecule is a whole antibody or ascAb.

In some embodiments, the antibody molecule comprises a payload which is cytotoxic.

A second aspect of the invention provides a pharmaceutical composition for use in a method of treatment of an Acinetobacter bacterial infection, the composition comprising an antibody molecule as defined in any embodiment of the first aspect of the invention, and a pharmaceutically acceptable excipient.

A third aspect of the invention provides a composition of matter for use in a method of treatment of an Acinetobacter bacterial infection, the composition comprising (1) a pharmaceutical composition as defined in the second aspect of the invention and (2) a further antibacterial agent.

A fourth aspect of the invention provides a method of identifying or labelling an Acinetobacter cell, the method comprising contacting the cell with any suitable antibody molecule as defined in any embodiment of the first aspect of the invention.

A fifth aspect of the invention provides a method of opsonising, or increasing the rate of opsonisation of, an Acinetobacter cell, the method comprising contacting or pre-incubating the cell with any suitable antibody molecule as defined in any embodiment of the first aspect of the invention.

A sixth aspect of the invention provides a method of increasing the rate of engulfment of an Acinetobacter cell, the method comprising contacting the cell with any suitable antibody molecule as defined in any embodiment of the first aspect of the invention.

A seventh aspect of the invention provides a method of treatment of an Acinetobacter bacterial infection, comprising administering an antibody molecule as defined in any embodiment of the first aspect of the invention, or a composition as defined in any embodiment of the second of third aspect of the invention, to an individual in need thereof.

In some embodiments, the Acinetobacter bacterial infection is an A. baumannii bacterial infection.

In some embodiments, the Acinetobacter bacterial infection is an A. pittii infection, an A. nosocomialis infection, an A. calcoaceticus infection, an A. seifertii infection or an A. dijkshoorniae infection.

In some embodiments, the treatment comprises administering a second antibacterial agent, wherein the second antibacterial agent is optionally a cephalosporin, a combination beta-lactam/beta-lactamase inhibitor (optionally sulbactam), carbapenem (optionally meropenem, doripenem, or imipenem), a polymyxin (optionally colistin or polymixin B), tigecycline or minocycline.

An eighth aspect of the invention provides a use of an antibody molecule as defined in any embodiment of the first aspect of the invention in the manufacture of a medicament for use in treating or preventing an Acinetobacter infection.

In some embodiments of the first, second, third, seventh or eighth aspect of the invention, the Acinetobacter bacterial infection is in an immunosuppressed individual.

A ninth aspect of the invention provides a method for detecting the presence or absence of a bacterium which is Acinetobacter spp, the method comprising

(i) contacting a sample suspected of containing the bacterium with any suitable antibody molecule as defined in any embodiment of the first aspect of the invention, and

(ii) determining whether the antibody molecule binds to the sample, wherein binding of the antibody molecule to the sample indicates the presence of the bacterium.

A tenth aspect of the invention provides a method for diagnosing a bacterial infection in an individual which is caused by Acinetobacter spp, the method comprising

(i) contacting a biological sample obtained from the individual with any suitable antibody molecule as defined in any embodiment of the first aspect of the invention, and

(ii) determining whether the antibody molecule binds to the biological sample, wherein binding of the antibody molecule to the biological sample indicates the presence of a bacterial infection.

An eleventh aspect of the invention provides a linear flow device (LFD) for detecting an analyte which is a bacterial pathogen in a sample fluid,
wherein said LFD comprises:

(i) a housing, and

(ii) at least one flow path leading from a sample well to a viewing window, wherein said flow path comprises one or more carriers along which the sample fluid is capable of flowing by capillary action, the or each carrier comprising an analyte-detecting means;
wherein the presence of analyte produces a line in the viewing window which indicates an analyte concentration,
wherein the bacterial pathogen is Acinetobacter spp. and the at least one analyte-detecting means is any suitable antibody molecule as defined in any embodiment of the first aspect of the invention.

In some embodiments, the device comprises a control zone capable of indicating the assay has been successfully run.

In some embodiments, the device comprises a plurality of analyte-detecting means capable of distinguishing between multiple bacterial pathogens, wherein one of the analyte-detecting means is any suitable antibody molecule as defined in any embodiment of the first aspect of the invention.

In some embodiments, the multiple bacterial pathogens comprise A. baumannii, plus one or more or all of Pseudomonas aeruginosa, Escherichia coli and Serratia marcescens.

In some embodiments, the multiple bacterial pathogens comprise A. pittii, A. nosocomialis, A. calcoaceticus, A. seifertii or A. dijkshoorniae, plus one or more or all of Pseudomonas aeruginosa, Escherichia coli and Serratia marcescens.

A twelfth aspect of the invention provides any embodiment of the eleventh aspect of the invention for use in any embodiment of the seventh aspect of the invention.

Some of these aspects and embodiments of the invention will now be described in more detail.

Production of the antibodies of the invention is described in WO2016/142660, the disclosure of which is specifically incorporated herein by cross-reference.

mAbs and Processes of Production

Pooled immunoglobulin from serum was one of the first widely available treatments for microbial infections and that hyperimmune human sera immunoglobulin is still used today to treat a number of infections including cytomegalovirus (CMV), hepatitis A and B virus (HAV, HBV) rabies and measles (12-14). Nevertheless, although in recent years humanised versions of mAbs have become some of the world's bestselling drugs, to date the majority of these mAbs have been licensed for the treatment of cancer and autoimmune diseases (9-11), there is currently only one mAb approved for the treatment of an infectious disease (13).

Methods for the production of mAbs for therapeutic and/or diagnostic use have diversified dramatically over the decades. Early mAbs were mainly of murine origin but tended to be immunogenic in the human host (34, 35). The majority of mAbs currently in the clinics are humanized or fully human IgG1 mAbs generated through hybridoma cell lines (14, 35). Combinatorial display technologies using phage or yeast have been valuable but require a period of in vitro affinity maturation and lose the natural antibody heavy and light chain pairings (14).

Recently, direct amplification of individual VH and VL chain domain genes from single human B cells to ensure retention of native antibody heavy and light chain pairings, has led to the generation of fully human mAbs with increased safety and relevance to human disease in areas where current treatments are suboptimal (14, 36-39).

Antibody Molecules

Anti-Candida recombinant human antibody molecules of the invention may include any polypeptide or protein comprising an antibody antigen-binding site described herein, including Fab, Fab₂, Fab₃, diabodies, triabodies, tetrabodies, minibodies and single-domain antibodies, as well as whole antibodies of any isotype or sub-class.

The anti-Candida recombinant human antibody molecules may also be a single-chain variable fragment (scFv) or single-chain antibody (scAb). An scFv fragment is a fusion of a variable heavy (VH) and variable light (VL) chain. A scAb has a constant light (CL) chain fused to the VL chain of an scFv fragment. The CL chain is optionally the human kappa light chain (HuCK). A single chain Fv (scFv) may be comprised within a mini-immunoglobulin or small immunoprotein (SIP), e.g. as described in Li et al. (1997). An SIP may comprise an scFv molecule fused to the CH4 domain of the human IgE secretory isoform IgE-S2 (ϵ_{S2}-CH4; Batista, F. D., Anand, S., Presani, G., Efremov, D. G. and Burrone, O. R. (1996). The two membrane isoforms of human IgE assemble into functionally distinct B cell antigen receptors. J. Exp. Med. 184:2197-2205) forming an homo-dimeric mini-immunoglobulin antibody molecule.

Antibody molecules and methods for their construction and use are described, in for example, Holliger, P. and Hudson, P. J. (2005). Engineered antibody fragments and the rise of single domains. Nat. Biotechnol. 23:1126-1136.

Anti-Candida recombinant human antibody molecules as described herein may lack antibody constant regions.

However in some preferred embodiments, the anti-Candida recombinant human antibody molecule is a whole antibody. For example, the anti-Candida recombinant human antibody molecule may be an IgG, IgA, IgE or IgM or any of the isotype sub-classes, particularly IgG1.

Anti-Candida recombinant human antibody molecules as described will generally be provided in isolated form, in the sense of being free from contaminants, such as antibodies able to bind other polypeptides and/or serum components.

Anti-Candida recombinant human antibody molecules of the invention may be obtained in the light of the disclosure herein, for example using techniques described in reference (14).

Antibody molecules of the invention typically comprise an antigen binding domain comprising an immunoglobulin heavy chain variable domain (VH) and an immunoglobulin light chain variable domain (VL).

Each of the VH and VL domains typically comprise 3 complementarity determining regions (CDRs) responsible for antigen binding, interspersed by 4 framework (FW) regions.

In Tables C-F hereinafter, the sequences of each of the CDRs and FWs for each of the VH and VL domains is given for each of the preferred 4 antibodies of the invention i.e. Antibodies 120-123 (directed to the Hyr1 protein).

Tables VH and VL give the entire VH and VL domains of these 4 antibodies.

In these tables, each antibody the sequences are numbered as follows:

| | |
|---|---|
| 1x | H FW1 |
| 2x | H CDR1 |
| 3x | H FW2 |
| 4x | H CDR2 |
| 5x | H FW3 |
| 6x | H CDR3 |
| 7x | H FW4 |
| 8x | L FW1 |
| 9x | L CDR1 |
| 10x | L FW2 |
| 11x | L CDR2 |
| 12x | L FW3 |
| 13x | L CDR3 |
| 14x | L FW4 |
| 15x | VH full sequence |
| 16x | VL full sequence |

In each case ‘x’ represents any single letter of C-F, each letter representing one of the 4 antibodies 120-123; for example ‘F’ represents antibody AB123 described in Table F. It will be understood that the description in relation to sequence ‘x’ applies mutatis mutandis to any of the antibodies described in Tables C-F, as if that description was written individually for each antibody.

In some embodiments, Anti-Candida recombinant human antibody molecules bind to the target wholly or substantially through a VHCDR3 sequence described herein.

Substitutions as described herein may be conservative substitutions or may be present to remove Cys residues from the native sequence. In some embodiments, an antibody may comprise one or more substitutions, deletions or insertions which remove a glycosylation site.

The HCDR3 may be the only region of the antibody molecule that interacts with a target epitope or substantially the only region. The HCDR3 may therefore determine the specificity and/or affinity of the antibody molecule for the target.

The VH domain of an anti-Candida recombinant human antibody molecule may additionally comprise an HCDR2 having the amino acid sequence of SEQ ID NO: 4x or the sequence of SEQ ID NO: 4x with 1 or more, for example 2, or 3 or more amino acid substitutions, deletions or insertions.

The VH domain of an anti-Candida recombinant human antibody molecule may further comprise an HCDR1 having the amino acid sequence of SEQ ID NO: 2x or the sequence of SEQ ID NO: 2x with 1 or more, for example 2 or 3 or more amino acid substitutions, deletions or insertions.

In some embodiments, an antibody molecule may comprise a VH domain comprising a HCDR1, a HCDR2 and a HCDR3 having the sequences of SEQ ID NOs 2x, 4x and 6x respectively.

In some embodiments, an antibody molecule may comprise a VH domain comprising one or more or all of a FW1, a FW2, a FW3 and a FW4 having the sequences of SEQ ID NOs lx, 3x, 5x and 7x respectively. Any of these FW regions may include 1 or more, for example 2 or 3 or more amino acid substitutions, deletions or insertions.

For example, an antibody molecule may comprise a VH domain having the sequence of SEQ ID NO: 15x or the sequence of SEQ ID NO: 15x with 1 or more, for example 2, 3, 4, 5, 6, 7, 8, 9, 10 or more amino acid substitutions, deletions or insertions in SEQ ID NO: 15x.

The anti-Candida recombinant human antibody molecule will typically further comprise a VL domain, for example a VL domain comprising LCDR1, LCDR2 and LCDR3 having the sequences of SEQ ID NOs 9x, 11x and 13x respectively, or the sequences of SEQ ID NOs 9x, 11x and 13x respectively with, independently, 1 or more, for example 2 or 3 or more amino acid substitutions, deletions or insertions.

In some embodiments, an antibody molecule may comprise a VL domain comprising one or more or all of a FW1, a FW2, a FW3 and a FW4 having the sequences of SEQ ID NOs 8x, 10x, 12x and 14x respectively. Any of these may include 1 or more, for example 2 or 3 or more amino acid substitutions, deletions or insertions.

For example, an antibody molecule may comprise a VL domain having the sequence of SEQ ID NO: 16x or the sequence of SEQ ID NO: 16x with 1 or more, for example 2, 3, 4, 5, 6, 7, 8, 9, 10 or more amino acid substitutions, deletions or insertions in SEQ ID NO: 16x.

The anti-Candida recombinant human antibody molecule may for example comprise one or more amino acid substitutions, deletions or insertions which improve one or more properties of the antibody, for example affinity, functional half-life, on and off rates.

The techniques required in order to introduce substitutions, deletions or insertions within amino acid sequences of CDRs, antibody VH or VL domains and antibodies are generally available in the art. Variant sequences may be made, with substitutions, deletions or insertions that may or may not be predicted to have a minimal or beneficial effect on activity, and tested for ability to bind to C. albicans antigens and/or for any other desired property.

In some embodiments, an anti-Candida recombinant human antibody molecule may comprise a VH domain comprising a HCDR1, a HCDR2 and a HCDR3 having the sequences of SEQ ID NOs 2x, 4x, and 6x, respectively, and a VL domain comprising a LCDR1, a LCDR2 and a LCDR3 having the sequences of SEQ ID NOs 9x, 11x and 13x, respectively.

For example, the VH and VL domains may have the amino acid sequences of SEQ ID NO: 15x and SEQ ID NO: 16x respectively; or may have the amino acid sequences of SEQ ID NO: 15x and SEQ ID NO: 16x comprising, independently 1 or more, for example 2, 3, 4, 5, 6, 7, 8, 9, 10 or more amino acid substitutions, deletions or insertions.

In some embodiments, an anti-Candida recombinant human antibody molecule VH domain may have at least about 60% sequence identity to SEQ ID NO: 15x, e.g. at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 15x.

In some embodiments, an anti-Candida recombinant human antibody molecule VL domain may have at least about 60% sequence identity to SEQ ID NO: 16x, e.g. at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 16x.

The anti-Candida recombinant human antibody molecule may be in any format, as described above, In some preferred embodiments, the anti-Candida recombinant human antibody molecule may be a whole antibody, for example an IgG, such as IgG1, IgA, IgE or IgM. In some preferred embodiments, than anti-Candida recombinant human antibody molecule is a scAb or scFv.

An anti-Candida recombinant human antibody molecule may be one which competes for binding to the target (e.g. Hyr1) with an antibody molecule described herein, for example an antibody molecule which

(i) binds Hyr1 and

(ii) comprises a VH domain of SEQ ID NO: 15x and/or VL domain of SEQ ID NO: 16x; an HCDR3 of SEQ ID NO: 6x; an HCDR1, HCDR2, LCDR1, LCDR2, or LCDR3 of SEQ ID NOS: 2x, 4x, 9x, 11x or 13x respectively; a VH domain comprising HCDR1, HCDR2 and HCDR3 sequences of SEQ ID NOS: 2x, 4x and 6x respectively; and/or a VH domain comprising HCDR1, HCDR2 and HCDR3 sequences of SEQ ID NOS: 2x, 4x and 6x and a VL domain comprising LCDR1, LDR2 and LCDR3 sequences of SEQ ID NOS: 9x, 11x and 13x respectively,

where x here is C, D, E, or F.

Competition between antibody molecules may be assayed easily in vitro, for example using ELISA and/or by tagging a specific reporter molecule to one antibody molecule which can be detected in the presence of one or more other untagged antibody molecules, to enable identification of antibody molecules which bind the same epitope or an overlapping epitope. Such methods are readily known to one of ordinary skill in the art.

Thus, a further aspect of the present invention provides a binding member or antibody molecule comprising an antigen-binding site that competes with an antibody molecule, for example an antibody molecule comprising a VH and/or VL domain, CDR e.g. HCDR3 or set of CDRs of the parent antibody described above for binding to target antigen. A suitable antibody molecule may comprise an antibody antigen-binding site which competes with an antibody antigen-binding site for binding to target antigen wherein the antibody antigen-binding site is composed of a VH domain and a VL domain, and wherein the VH and VL domains comprise HCDR1, HCDR2 and HCDR3 sequences of SEQ ID NOS: 2x, 4x, and 6x and LCDR1, LDR2 and LCDR3 sequences of SEQ ID NOS: 9x, 11x, and 13x respectively, for example the VH and VL domains of SEQ ID NOS: 15x and 16x.

The VH and VL framework encoded by the genes encoded from the B cell antibody factories can be readily modified by molecular genetics to alter and refine the properties of the antibodies. Such modified sequences are termed “derived” from the B cells herein.

For example it may be desired to remove Cys residues in the sequence, to minimise potential incorrect Cys pairings.

Suitable techniques for the maturation and optimisation of antibody molecules are well-known in the art.

Anti-Candida recombinant human antibody molecules may be further modified by chemical modification, for example by PEGylation, or by incorporation in a liposome, to improve their pharmaceutical properties, for example by increasing in vivo half-life.

An anti-Candida recombinant human antibody molecule as described herein may be conjugated to a toxic payload (e.g. ricin) that could kill Acinetobacter bacteria and act as a therapeutic antibody.

An anti-Candida recombinant human antibody molecule as described herein may be one which binds A. baumannii with an EC₅₀ values of 1 to 1500, e.g. 1 to 500 or 1 to 40 ng/ml, or less than 1400 ng/mL, less than 1300 ng/mL, less than 1200 ng/mL, less than 1100 ng/mL, less than 1000 ng/mL, less than 900 ng/mL, less than 800 ng/mL, less than 700 ng/mL, less than 600 ng/mL, less than 500 ng/mL, less than 400 ng/mL, less than 300 ng/mL, less than 200 ng/mL, less than 100 ng/mL, less than 90 ng/mL, less than 80 ng/mL, less than 70 ng/mL, less than 60 ng/mL, less than 50 ng/mL, less than 40 ng/mL, less than 30 ng/mL, less than 20 ng/mL, less than 10 ng/mL, less than 5 ng/mL, less than 4 ng/mL, less than 3 ng/mL, less than 2 ng/mL or less than 1 ng/mL.

EC₅₀ can be assessed as described hereinafter with ELISA e.g. as described in the

Examples below for “Circulating IgG Enzyme-linked Immunosorbent assay (ELISA) to identify donors with B cells to take forward ” and “B cell supernatant screen against target antigens via ELISA”.

It is known that there are a large number of Acinetobacter species. Key Acinetobacter species which may be targeted by the antibodies described herein include members of the Acinetobacter baumannii-Acinetobacter calcoaceticus complex, which comprises A. baumannii, A. pittii, A. nosocomialis, A. calcoaceticus, A. seifertii and A. dijkshoorniae.

As described herein, the anti-Candida recombinant human antibody molecules of the invention can detect both morphology-specific and morphology-independent epitopes with high specificity. The antibody molecules described herein may thus bind to A. baumannii with high affinity relative to other bacterial targets. For example, an antibody molecule of the invention may display a binding affinity for A. baumannii which is at least 1000 fold or at least 2000 fold greater than non-Acinetobacter pathogenic bacteria such as Pseudomonas aeruginosa, Escherichia coli and Serratia marcescens.

Nevertheless an anti-Candida recombinant human antibody molecule as described herein may bind to species closely related to A. baumannii e.g. members of the Acinetobacter baumannii-Acinetobacter calcoaceticus complex, which comprises A. baumannii, A. pittii, A. nosocomialis, A. calcoaceticus, A. seifertii and A. dijkshoomiae, for example with an affinity within a 1000-fold of the binding to A. baumannii (assessed using EC₅₀).

Provided herein is a method of opsonising, or increasing the rate of opsonisation of an Acinetobacter cell, for example A. baumannii, the method comprising contacting or pre-incubating the Acinetobacter cell with an anti-Candida recombinant human antibody molecule as described herein.

Provided herein is a method of increasing the rate of engulfment of a Acinetobacter cell, for example A. baumannii, by macrophages, the method comprising contacting the Acinetobacter cell with an anti-Candida recombinant human antibody molecule as described herein.

Treatment of Disease

An anti-Candida recombinant human antibody molecule as described herein may be used for clinical benefit in the treatment of an Acinetobacter-associated condition, and particularly infections caused by A. baumannii species.

An anti-Candida recombinant human antibody molecule as described herein may also be used for clinical benefit in the treatment of an Acinetobacter-associated condition caused by an A. pittii infection, an A. nosocomialis infection, an A. calcoaceticus infection, an A. seifertii infection or an A. dijkshoomiae infection.

The antibody molecules as described herein may be useful in the surgical and other medical procedures which may lead to immunosuppression, or medical procedures in patients who are already immunosuppressed.

Patients suitable for treatment as described herein include patients with conditions in which Acinetobacter infection is a symptom or a side-effect of treatment or which confer an increased risk of Acinetobacter infection or patients who are predisposed to or at increased risk of Acinetobacter infection, relative to the general population. For example, an anti-Candida recombinant human antibody molecule as described herein may also be useful in the treatment or prevention of Acinetobacter infection in cancer patients.

An anti-Candida recombinant human antibody molecule as described herein may be used in a method of treatment of the human or animal body, including prophylactic or preventative treatment (e.g. treatment before the onset of a condition in an individual to reduce the risk of the condition occurring in the individual; delay its onset; or reduce its severity after onset). The method of treatment may comprise administering an anti-Candida recombinant human antibody molecule to an individual in need thereof.

Pharmaceutical Compositions and Dosage Regimens

Anti-Candida recombinant human antibody molecules may be comprised in pharmaceutical compositions with a pharmaceutically acceptable excipient.

A pharmaceutically acceptable excipient may be a compound or a combination of compounds entering into a pharmaceutical composition which does not provoke secondary reactions and which allows, for example, facilitation of the administration of the anti-Candida recombinant human antibody molecule, an increase in its lifespan and/or in its efficacy in the body or an increase in its solubility in solution. These pharmaceutically acceptable vehicles are well known and will be adapted by the person skilled in the art as a function of the mode of administration of the anti-Candida recombinant human antibody molecule.

In some embodiments, anti-Candida recombinant human antibody molecules may be provided in a lyophilised form for reconstitution prior to administration. For example, lyophilised antibody molecules may be re-constituted in sterile water and mixed with saline prior to administration to an individual.

Anti-Candida recombinant human antibody molecules will usually be administered in the form of a pharmaceutical composition, which may comprise at least one component in addition to the antibody molecule. Thus pharmaceutical compositions may comprise, in addition to the anti-Candida recombinant human antibody molecule, a pharmaceutically acceptable excipient, carrier, buffer, stabiliser or other materials well known to those skilled in the art. Such materials should be non-toxic and should not interfere with the efficacy of the anti-Candida recombinant human antibody molecule. The precise nature of the carrier or other material will depend on the route of administration, which may be by bolus, infusion, injection or any other suitable route, as discussed below.

For parenteral, for example sub-cutaneous or intra-venous administration, e.g. by injection, the pharmaceutical composition comprising the anti-Candida recombinant human antibody molecule may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilisers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc, is within the responsibility of general practitioners and other medical doctors and may depend on the severity of the symptoms and/or progression of a disease being treated. Appropriate doses of antibody molecules are well known in the art (Ledermann, J. A., Begent, R. H., Massof, C., Kelly, A. M., Adam, T. and Bagshawe, K. D. (1991). A phase-I study of repeated therapy with radiolabelled antibody to carcinoembryonic antigen using intermittent or continuous administration of cyclosporin A to suppress the immune response. Int. J. Cancer 47:659-664). Specific dosages may be indicated herein or in the Physician's Desk Reference (2003) as appropriate for the type of medicament being administered may be used. A therapeutically effective amount or suitable dose of an antibody molecule may be determined by comparing it's in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g. whole antibody, fragment) and the nature of any detectable label or other molecule attached to the antibody.

A typical antibody dose will be in the range 100 μg to 1 g for systemic applications, and 1 μg to 1 mg for topical applications. An initial higher loading dose, followed by one or more lower doses, may be administered.

Typically, the antibody will be a whole antibody, e.g. the IgG1 isotype, and where a whole antibody is used, dosages at the lower end of the ranges described herein may be preferred. This is a dose for a single treatment of an adult patient, which may be proportionally adjusted for children and infants, and also adjusted for other antibody formats in proportion to molecular weight.

Preferably the antibody or fragment will be dosed at no more than 50 mg/kg or no more than 100 mg/kg in a human patient, for example between 1 and 50, e.g. 5 to 40, 10 to 30, 10 to 20 mg/kg.

Treatments may be repeated at daily, twice-weekly, weekly or monthly intervals, at the discretion of the physician. The treatment schedule for an individual may be dependent on the pharmocokinetic and pharmacodynamic properties of the antibody composition, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, anti-Candida recombinant human antibody molecules as described herein may be administered as sub-cutaneous injections. Sub-cutaneous injections may be administered using an auto-injector, for example for long term prophylaxis/treatment.

In some preferred embodiments, the therapeutic effect of the anti-Candida recombinant human antibody molecule may persist for several half-lives, depending on the dose. For example, the therapeutic effect of a single dose of anti-Candida recombinant human antibody molecule may persist in an individual for 1 month or more, 2 months or more, 3 months or more, 4 months or more, 5 months or more, or 6 months or more.

Combination Immunotherapy

It will be understood that the term “treatment” as used herein includes combination treatments and therapies, in which two or more treatments, therapies, or agents are combined, for example, sequentially or simultaneously.

The agents (i.e. the anti-Candida recombinant human antibody molecules described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g. 1, 2, 3, 4 or more hours apart, or even longer periods apart where required), the precise dosage regimen being commensurate with the properties of the therapeutic agent(s) as described herein, including their synergistic effect.

The agents (i.e. the anti-Candida recombinant human antibody molecules described here, plus one or more other agents) may be formulated together in a single dosage form, or alternatively, the individual agents may be formulated separately and presented together in the form of a kit, optionally with instructions for their use.

For example, the compounds described herein may in any aspect and embodiment also be used in combination therapies, e.g. in conjunction with other agents e.g. antibacterial agents. The second antibacterial agent may be selected from a cephalosporin, a combination beta-lactam/beta-lactamase inhibitor (optionally sulbactam), carbapenem (optionally meropenem, doripenem, or imipenem), a polymyxin (optionally colistin or polymixin B), tigecycline or minocycline.

The skilled person will recognise that other antibacterial agents may also be used. In some embodiments, the second antibacterial agent is a second anti-bacterial antibody or an antimicrobial peptide. In some embodiments, the anti-Candida recombinant human antibody molecule described herein is conjugated to the second antibacterial agent.

Preparation of Other Therapeutic Moieties

The anti-Candida recombinant human antibody molecules described herein may be utilised to isolate and identify protective antigens for development as Acinetobacter vaccines, or prepare or identify other therapeutic moieties.

For example the antigens bound by the mAbs described herein may be identified by methods known to those skilled in the art. For example they could be screened against protein and carbohydrate mutants to identify those mutants where binding is reduced. Alternatively antigens can be identified more directly by a proteomics-based approach, for example using 2D electrophoresis and immunoblotting, followed by analysis of spots by trypsinization and mass-spectroscopy (see e.g. Silva et al. Mol Biochem Parasitol. 2013 April; 188(2):109-15.). Such antigens will have utility as potential vaccines.

Anti-idiotype antibodies can be prepared to the antibodies described herein using methods well known to those in the art (see Polonelli, L et al. “Monoclonal Yeast Killer Toxin-like Candidacidal Anti-Idiotypic Antibodies.” Clinical and Diagnostic Laboratory Immunology 4.2 (1997): 142-146; also U.S. Pat. No. 5,233,024).

Detection and Diagnosis

Anti-Candida recombinant human antibody molecules as described herein may also be useful in in vitro testing, for example in the detection of Acinetobacter bacteria or an Acinetobacter infection, for example in a sample obtained from a patient.

Anti-Candida recombinant human antibody molecules as described herein may be useful for identifying A. baumannii, and/or distinguishing A. baumanii from other bacteria.

Anti-Candida recombinant human antibody molecules as described herein may be useful for identifying A. pittii, A. nosocomialis, A. calcoaceticus, A. seifertii or A. dijkshoorniae, and/or distinguishing A. pittii, A. nosocomialis, A. calcoaceticus, A. seifertii or A. dijkshoomiae from other bacteria.

The presence or absence of an Acinetobacter bacterium (e.g. A. baumanni) may be detected by

(i) contacting a sample suspected of containing the bacterium with any suitable antibody molecule described herein, and

(ii) determining whether the antibody molecule binds to the sample, wherein binding of the antibody molecule to the sample indicates the presence of the bacterium.

An Acinetobacter infection, e.g. A. baumannii infection, in an individual may be diagnosed by

(i) obtaining a sample from the individual;

(ii) contacting the sample with any suitable antibody molecule as described herein, and

(iii) determining whether the antibody molecule binds to the sample, wherein binding of the antibody molecule to the sample indicates the presence of the Acinetobacter infection.

Binding of antibodies to a sample may be determined using any of a variety of techniques known in the art, for example ELISA, immunocytochemistry, immunoprecipitation, affinity chromatography, and biochemical or cell-based assays. In some embodiments, the antibody is conjugated to a detectable label or a radioisotope.

Lateral Flow Devices

The invention also provides rapid and highly specific diagnostic tests for detecting Acinetobacter pathogens, for example multiple bacterial pathogens, in a single test.

Preferred tests detect not only A. baumannii, but also one or more other major Acinetobacter pathogens e.g. members of the Acinetobacter baumannii-Acinetobacter calcoaceticus complex, which comprises A. baumannii, A. pittii, A. nosocomialis, A. calcoaceticus, A. seifertii and A. dijkshoorniae. Antibody molecules specific for these other pathogens may be provided in the light of the disclosure herein, for example.

Preferably the test is in the form of a lateral flow device (LFD). Such LFDs are particularly suitable for use as point-of-care Acinetobacter diagnostics.

A lateral flow assay device for the analysis of body fluid will comprise at its most basic:

(i) a housing, and

(ii) a flow path.

The devices, systems and methods described herein are for measuring analyte levels in body fluids of animals, particularly mammals including humans, or in environmental samples e.g. where it is believed Acinetobacter pathogens may exist.

As used anywhere herein, unless context demands otherwise, the term ‘body fluid’ may be taken to mean any fluid found in the body of which a sample can be taken for analysis. Examples of body fluids suitable for use in the present invention include, but are not limited to blood, urine, sweat and saliva. Preferably, the body fluid is blood. The fluid may be diluted by a pre-determined amount prior to assay, and any quantification indicator on the

LFD may reflect that pre-determined dilution.

Some aspects of the LFD will now be discussed in more detail:

Flow Path of LFD

The flow path (e.g. a chromatographic strip) is preferably provided by a carrier, through which the test substance or body fluid can flow by capillary action. In one embodiment, the carrier is a porous carrier, for example a nitrocellulose or nylon membrane. In a further embodiment, sections or all of the carrier may be non-porous. For example, the non-porous carrier may comprise areas of perpendicular projections (micropillars) around which lateral capillary flow is achieved, as described in for example WO2003/103835, WO2005/089082 and WO2006/137785, incorporated herein by reference.

The flow path will typically have an analyte-detection zone comprising a conjugate release zone and a detection zone where a visible signal reveals the presence (or absence) of the analyte of interest. The test substance can be introduced into the LFD and flows through to the detection zone.

Preferably the carrier material is in the form of a strip, sheet or similar to the material described in WO2006/137785 to which the reagents are applied in spatially distinct zones. The body fluid sample is allowed to permeate through the sheet, strip or other material from one side or end to another.

Analyte Detection Methods

Analyte detection may be based on competitive or sandwich (non-competitive) assays. Such assays may be used to detect analytes (antigens) from A. baumannii, plus optionally one or more other major Acinetobacter pathogens e.g. from members of the Acinetobacter baumannii-Acinetobacter calcoaceticus complex, which comprises A. baumannii, A. pittii, A. nosocomialis, A. calcoaceticus, A. seifertii and A. dijkshoomiae.

The conjugate release zone may contain freely mobile antibodies to the analyte of interest. Alternatively, the conjugate release zone may comprise reagents for carrying out a particular assay to enable detection of the analyte, as described herein.

The binding partners may be attached to a mobile and visible label. A “label” is a composition detectable by spectroscopic, photochemical, biochemical, immunochemical, electrical, optical or chemical means. Useful labels in the present invention include magnetic beads (e.g., Dynabeads™), fluorescent dyes, radiolabels, enzymes, and colorimetric labels such as colloidal gold, silver, selenium, or other metals, or coloured glass or plastic (e.g., polystyrene, polypropylene, latex, etc.) beads. Preferred is a gold colloid or latex bead.

If the analyte is present in the sample, it will bind to the labelled binding partners. In preferred embodiments the intensity of the colour may be directly proportional to the amount of analyte. Here the detection zone comprises permanently immobilised unlabelled specific binding reagent for the same analyte. The relative positioning of the labelled binding partner and detection zone being such that a body fluid sample applied to the device can pick up labelled binding partner and thereafter permeate into the detection zone. The amount of bound label can be detected as a visible signal in the detection zone.

The label in the LFD will be quantifiable by conventional means or as described herein.

In one competitive format embodiment, the detection zone contains regions of immobile analyte-protein derivatives. These bind and immobilise any of the labelled binding partners not already bound by the analyte in the sample, producing a coloured line or stripe. In this case the amount of label bound in the detection zone (and hence the intensity of the coloured stripe) will be inversely proportional to the amount of analyte in the sample.

In another competitive format, a labelled analyte or analyte analogue may alternatively be provided and this is detected using immobilized specific binding partner (e. g. immobilized antibody specific for the analyte) in the detection zone.

In another competitive format, a labelled analyte or analyte analogue is provided along with a specific binding partner (e.g. an antibody specific for the analyte). The resulting mixture is conveyed to the detection zone presenting immobilized binding partner of the analyte or analyte analogue. The higher the amount of analyte in the sample, the higher the amount of free labelled analyte which leaves the conjugate release zone to be detected in the detection zone.

Control Zone

Preferably the LFD for use with the present invention contains a control zone, which may be located after the detection zone in the direction of sample flow, in which excess labelled binding partner binds to produce a visible signal showing that the test has been successfully run.

Alternatively or additionally, a control zone may be located before the detection zone in the direction of sample flow, indicating that enough sample has been collected to allow operation of the test.

In one embodiment, the control zone is used as a reference point for a reader (see below).

Multiplex Devices

In various aspects of the invention, the LFD may be capable of detecting two (or more) different analytes e.g. analytes (antigens) from A. baumannii, plus optionally from one or more or all of members of the Acinetobacter baumannii-Acinetobacter calcoaceticus complex, which comprises A. baumannii, A. pittii, A. nosocomialis, A. calcoaceticus, A. seifertii and A. dijkshoorniae.

A number of multiplex formats are known in LFDs.

For example, the flow path may comprise two or more carriers. The carriers may be positioned along the flow path consecutively. In use, body fluid would flow along each carrier sequentially.

In a further embodiment, two or more carriers may be positioned in the flow path in parallel. In use, body fluid would flow along each carrier simultaneously.

In one embodiment, two analytes are analysed using two distinct flow path e.g. the housing of the LFD houses the two flow paths.

In one embodiment, the analyte-detecting means may comprise a first binding reagent that specifically binds the analyte and a second binding reagent that specifically binds the analyte, wherein the first binding reagent is labelled and is movable through a carrier under the influence of a liquid by capillary flow and the second binding reagent is immobilised at a detection site in the flow path. The analyte-detecting means comprises a labelled, mobile antibody, specific for the analyte and an immobilised unlabelled antibody, specific for the analyte.

In one embodiment, the analyte-detecting means for each analyte may be positioned together on the carrier, but the specific analyte-binding reagent for each different analyte may comprise a different label. The different labels will be capable of being distinguished as described herein or by conventional means.

Alternatively, the analyte-detecting means for each analyte may be spatially distinct. The flow path in the ‘multiplexed’ LFD may incorporate two or more discrete carriers of porous or non-porous solid phase material, e.g. each carrying mobile and immobilised reagents. These discrete bodies can be arranged in parallel, for example, such that a single application of body fluid sample to the device initiates sample flow in the discrete bodies simultaneously. The separate analytical results that can be determined in this way can be used as control results, or if different reagents are used on the different carriers, the simultaneous determination of a plurality of analytes in a single sample can be made. Alternatively, multiple samples can be applied individually to an array of carriers and analysed simultaneously.

Preferably, multiple analyte detection zones may be applied as lines spanning or substantially spanning the width of a test strip or sheet, preferably followed or preceded by one or more control zones in the direction of body fluid travel. However, multiple analyte detection zones may also, for example, be provided as spots, preferably as a series of discrete spots across the width of a test strip or sheet at the same height. In this case, a one or more control zones may again be provided after or before the analyte detection zones in the direction of body fluid travel.

Detection Systems

The presence or intensity of the signal in the detection zone may be determined by eye, optionally by comparison to a reference chart or card.

Where the intensity of the signal in the detection zone is to be converted to a quantitative reading of the concentration of analyte in the sample it will be preferred that the LFD can be used in conjunction with a screening device (reader). The reader is preferably a handheld electronic device into which the LFD cartridge can be inserted after the sample has been applied.

The reader comprises a light source such as an LED, light from which illuminates the LFD membrane. The reflected image of the membrane may be detected and digitised, then analysed by a CPU and converted to a result which can be displayed on an LCD screen or other display technology (or output via a conventional interface to further storage or analytical means). A light-dependent resistor, phototransistor, photodiode, CCD or other photo sensor may be used to measure the amount of reflected light. The result may be displayed as positive or negative for a particular analyte of interest or, preferably, the concentration of the particular analyte may be displayed. More specifically the conventional reader comprises: illuminating means for illuminating an immunoassay test; photosensitive detector means for detecting the intensity of light from the illuminating means which is reflected from the immunoassay test; means, coupled to the output of the photosensitive detector means, for representing the intensity of the detected light by a data array; memory means for storing preset data; first data processing means, coupled to the memory means and to the output of the means for representing the intensity of the detected light by a data array, for segmenting the data array according to the preset data into control data, background data and test data; second data processing means, coupled to the first data processing means, for determining whether the test data exhibits a statistically significant result; and output means, coupled to the output of the second data processing means, for outputting the results from the second data processing means.

In embodiments of the present invention where multiple analytes are assessed, the reader may analyse the results to detect a plurality of spatially distinct detection or test zones pertaining to different analytes. The photosensitive detector means (e.g. light dependent resistor, phototransistor, photodiode, CCD or other light sensor) will therefore detect reflected light from all of these (optionally scanning them) and generate a discrete or segmented data stream for each zone. Respective control zonal data and background zonal data may also be gathered for the different analytes.

The colour of the LED or other source may vary dependent on the label or method of detecting the analyte.

For gold-labelled analytes, a white LED may be preferable, and therefore a reader may comprise both a red and white LED.

Unless stated otherwise, or clear from the context, antibody residues, where numbered herein, are numbered in accordance with the Kabat numbering scheme.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1—Workflow for the generation of human monoclonal antibodies from single B cells. Class-switched memory B cells were isolated from individuals and microcultured in activating media to promote IgG secretion for screening against target antigens. VH and VL genes from B cells positive for the target were amplified and cloned into a mammalian expression vector for expression and purification via fast protein liquid chromatography. Following QC, recombinant mAbs were assessed for functional activity in vitro and in vivo. Adapted from Huang et al. 2013 (38).

FIG. 2—Representative images from the process employed to generate fully human anti-Candida mAbs. FIG. 2A shows the ELISA screening of purified donor circulating IgG against the antigen C. albicans ‘whole cell’ yeast and target antigen hyphae, and purified Hyr1 protein, to select the donors to take forward for B cell isolation. FIG. 2B and 2C are representative agarose gel images following RT-PCR and nested PCR of VH and Vk-Ck genes respectively. FIGS. 2D and 2E are analytical mass spectrometry and analytical SEC traces of one of the purified recombinant IgG1 mAbs. Further quality control was carried out by SDS-PAGE gel analysis under non-reducing and reducing conditions as shown in FIGS. 2F and 2G.

FIG. 3—Concentration response curves showing anti-Candida mAbs binding to target antigens. FIG. 3 shows purified anti-Hyr1 mAbs binding to purified recombinant Hyr1 protein in a concentration-dependent manner via ELISA. Values represent mean±SEM (n=2-4).

FIG. 4. Anti-Candida Hyr1 mAbs bind selectively to the gram negative bacteria, Acinetobacter baumannii. The anti-Hyr1 mAbs, AB120 (A), AB121 (B), AB122 (C), AB123 (D) and anti-Candida control AB as a negative control (E) were screened for their binding to whole cells of the gram negative bacteria: Acinetobacter baumannii, Escherichia coli, Serratia marcescens and Pseudomonas aeruginosa via ELISA. Values represent mean±SEM (n=4).

FIG. 5. Indirect immunofluorescence and TEM images of anti-CaHyr1 mAbs binding to A. baumannii cells. (A) Immunofluorescent images of anti-Hyr1 mAbs and negative control against A. baumannii using an alexa-488 conjugated secondary goat anti-human IgG antibody. (B) TEM images showing the ultrastructural binding of anti-Hyr1 mAbs to the cell surface of A. baumannii cells. A colloidal gold (10 nm) secondary goat anti-human IgG antibody was used to detect anti-Hyr1 mAb binding. Scale bars represent 10 μm on immunofluorescent images and 100 nm on TEM images.

FIG. 6. Treatment with anti-Hyr1 mAbs promote phagocytosis by macrophages and protects against A. baumannii infection. (A) Percentage of uptake events that occurred within the first 30 min of the assay following pre-incubation of A. baumannii with saline, an IgG control antibody or an anti-Hyr1 mAb. *p<0.005 compared to saline and control AB. (B) Images taken from live cell microscopy capturing images of phagocytosis of A. baumannii pre-incubated with 50 μg/ml control mAb or 50 μg/ml anti-Hyr1 mAb (AB123) at 30 min and 2 h.

FIG. 7. Treatment with anti-Hyr1 mAbs protects against A. baumannii infection. Galleria mellonella were infected with 1×10⁶ cells of A. baumannii following pre-incubation with (A) saline or 50 μg/ml anti-Hyr1 mAbs (B) AB120, (C) AB121, (D) AB122, (E) AB123, and their survival monitored every 12 h. *p<0.05, **p<0.01, ***p<0.005, ****p<0.0001, n =30.

FIG. 8—Indirect immunofluorescence of AB120 binding to Hyr1 protein expressed on C. albicans hyphal cells. Indirect immunofluorescence with anti-Hyr1 mAb AB120 against WT CAl4-Clp10 (A), Hyr1 null mutant (B) and a Hyr1 re-integrant strain (C). A fluorescently conjugated secondary goat anti-human IgG antibody was used to detect anti-Hyr1 mAb binding. Scale bars represent 15 μm.

FIG. 9—Schematic of VH, Vκ-Cκ and Vλ-Cλ cloning into pTT5 expression vector. B cells positive for antigen binding in the initial ELISA screen were lysed. mRNA in B cell lysate was used as a template for VH, Vκ-Cκ and Vλ-Cλ gene amplification via RT-PCR. RT-PCR was carried out using forward primers specific to human V domain leader sequences and reverse primers specific for human IgCH1, Cκ or Cλ regions or light chain UTR. To increase the specificity of gene amplification, nested PCR was carried out using RT-PCR products as the template. Forward primers specific for human VH FW1 sequences and reverse primers specific for human VH FW4 sequences were used to amplify VH genes. To capture Vκ-Cκ and Vλ-Cλ genes, forward primers specific to human Vκ and human Vλ FW1 sequences were used in combination with reverse primers specific to the 3′ end of the human Cκ or human Cλ regions. Primers used in nested PCR reactions contained 15 bp extensions which were complementary to the pTT5 expression vector to facilitate downstream Infusion cloning. Amplification of VH, Vκ-Cκ and Vλ-Cλ genes were done in separate reactions. RT-PCR—reverse transcriptase polymerase chain reaction; UTR untranslated region; L—leader sequence; V_{H}—heavy chain variable domain; Vκ—kappa chain variable domain; Vλ—lambda chain variable domain; C_{H}—heavy chain constant domain; Cκ—kappa chain constant domain; Cλ—lambda chain constant domain.

FIG. 10—Concentration response curves of purified anti-Hyr1 mAbs screened for binding to unrelated proteins. (a, b) Purified anti-Hyr1 mAbs screened against HSA and HEK NA respectively via ELISA. Values represent mean (n=2-4).

FIG. 11—Indirect immunofluorescence of mAbs binding to WT CAl4-Clp10 before and after enzymatic modification of the cell wall. Proteinase K treatment was used to reduce protein residues. Decrease in indirect immunofluorescence after enzymatic treatments suggested the nature of the mAb epitopes. Scale bars represent 4 μm.

FIG. 12. Treatment with anti-Hyr1 mAb prevents biofilm formation between C. albicans and A. baumannii. C. albicans was germinated in 10% serum for 2 h to form hyphae and then treated with either (A) 50 μg/ml of IgG1 control mAb or (B) 50 μg/ml of AB123 for 1 h, followed by co-incubation with A. baumannii for 3 h. Scale bars are 5 μm.

FIG. 13. Anti-Candida Hyr1 mAbs bind selectively to clinical isolates of Acinetobacter baumannii from different anatomical locations. The anti-Hyr1 mAb, AB123 and a control mAb were screened for their binding to whole cells of A. baumannii isolated from (A) blood, (B) respiratory samples, (C) tissues samples, (D) urine and (E) wound samples.

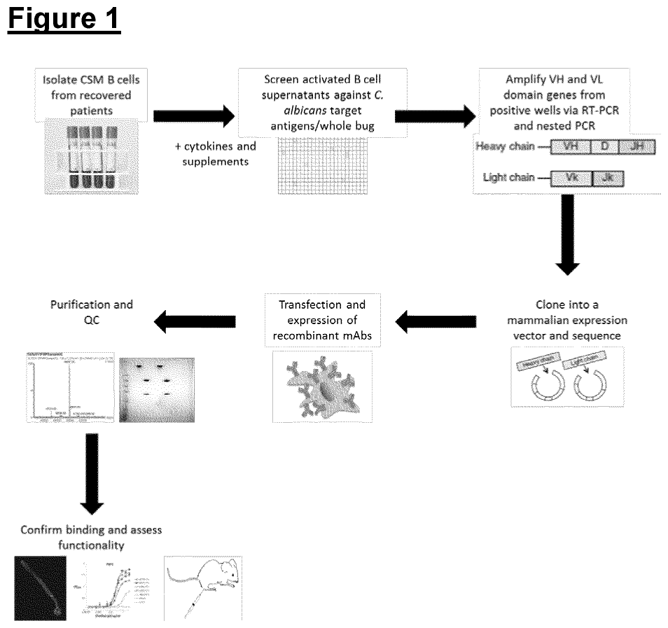

### EXAMPLES

### Example 1

### Generation of Fully Human Anti-Candida mAbs by Single B Cell Cloning

The generation of recombinant mAbs through direct amplification of VH and VL genes from single B cells produces fully human, affinity matured mAbs with the native antibody heavy and light chain pairing intact (14). We employed this technology to generate human recombinant anti-Candida mAbs to a defined C. albicans antigen—the morphogenesis-regulated protein 1 (Hyr1) protein expressed only in the hyphal cell wall (40).

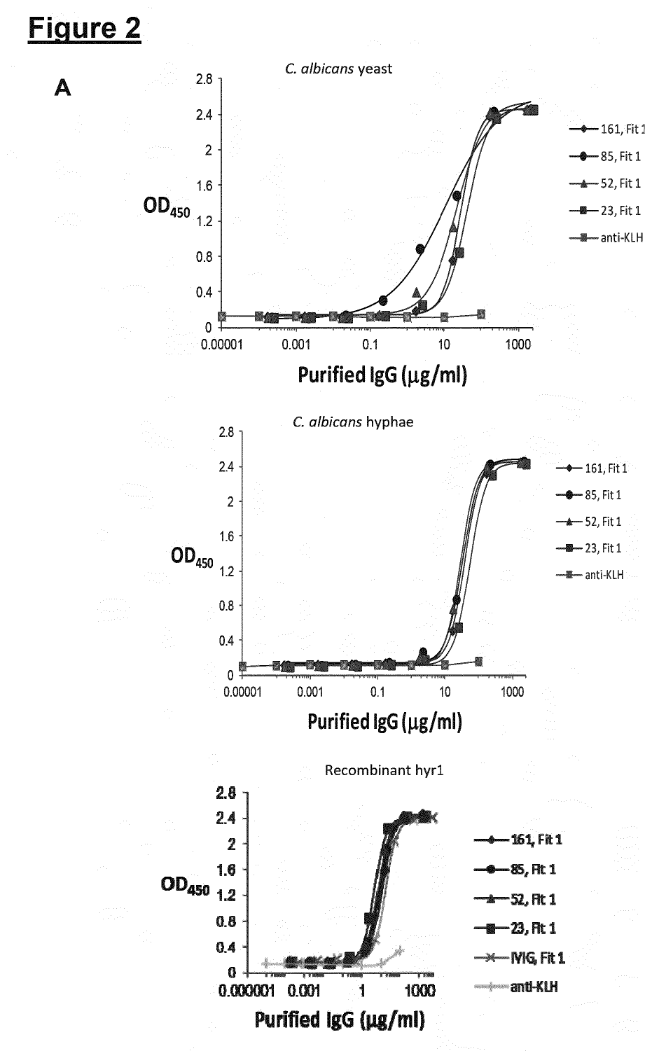

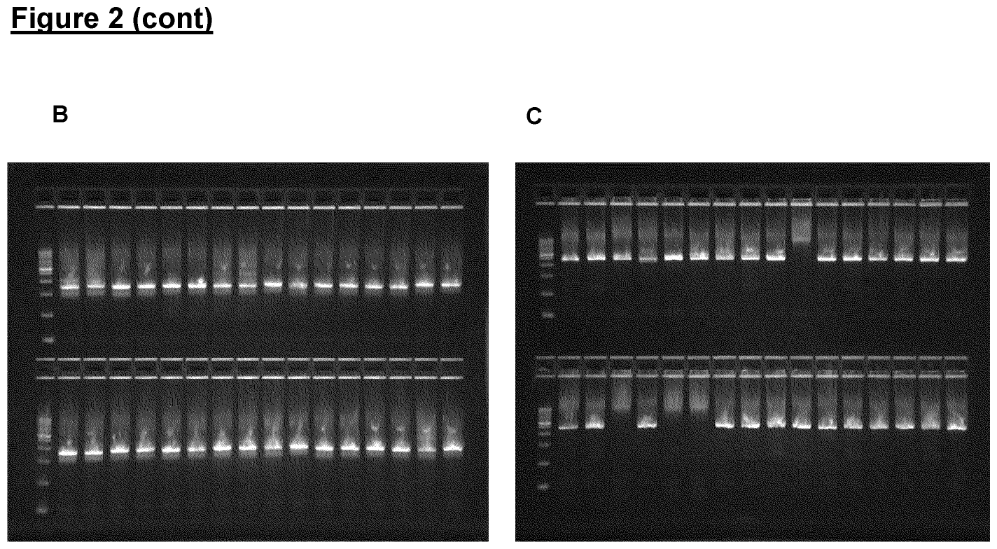

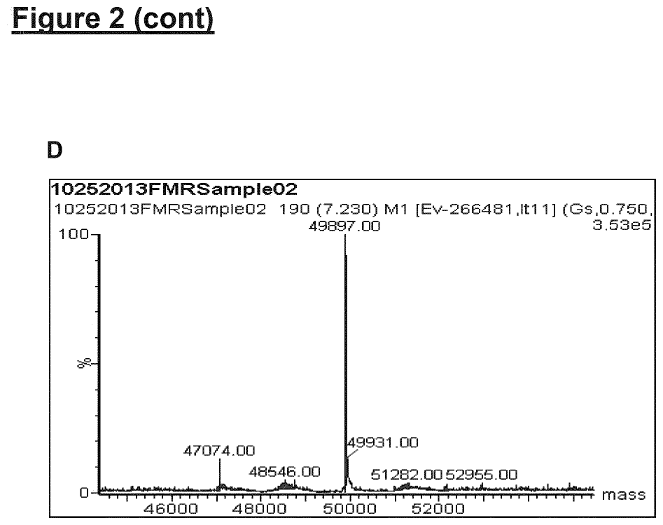

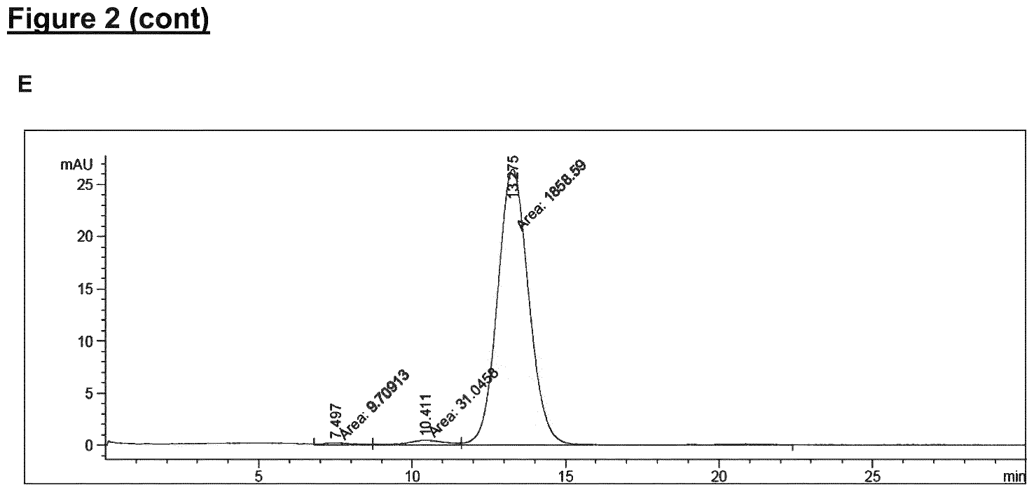

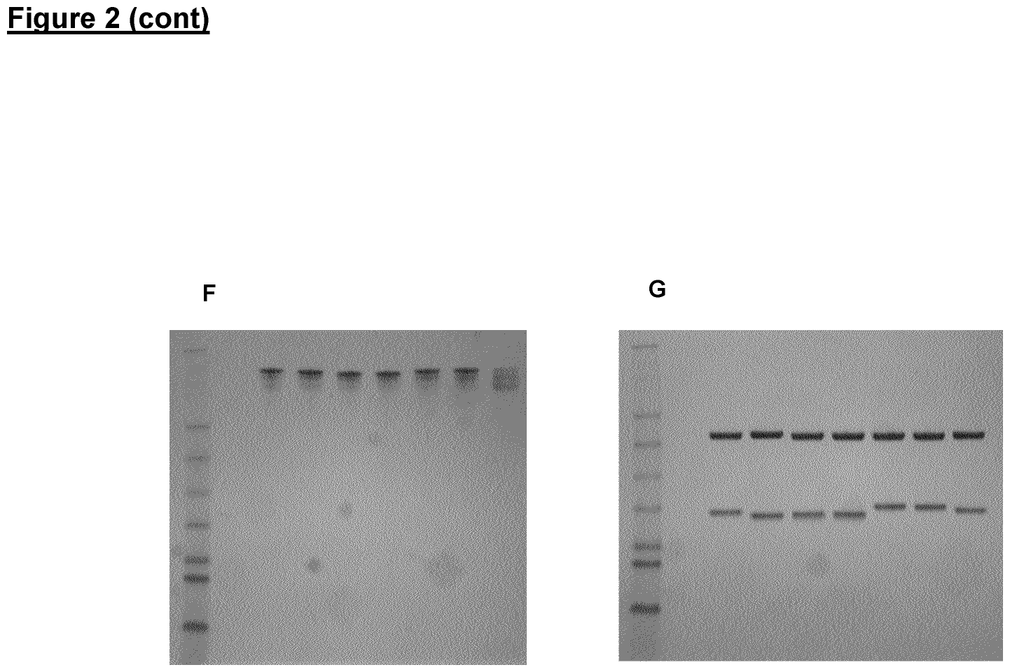

To enhance the likelihood of isolating Candida-related antibodies, the class switched memory (CSM) B cells used in this study were isolated from the blood of individuals who had recovered from a superficial Candida infection within a year of sampling. Donors were selected from a panel of volunteers and the levels of target-specific circulating IgG in the donor plasma was assessed via ELISA. In this screen, donor 23 had the highest IgG titre against Hyr1 (FIG. 2A). This donor was selected to provide the source of B cells to use for the generation of Candida-specific recombinant antibodies. After the isolation of CSM B cells from a donor, approximately 80000-150000 cells were plated out at 5 cells/well and activated with a cocktail of cytokines and supplements to promote secretion of IgG into the supernatant. A high throughput screening platform was then employed to facilitate the detection of IgG in the B cell supernatant against target antigens by ELISA. Positive ELISA hits enabled identification of wells containing B cells secreting antigen-specific IgG into the supernatant. Typically, approximately 0.05% wells/screen were positive (OD>4×background). Non-specific hits were identified and eliminated by performing an ELISA screen against two unrelated proteins—human serum albumin (HSA) and human embryonic kidney nuclear antigen (HEK NA). CSM B cells from wells that were positive for the antigen screen and negative for the unrelated protein screen were then lysed and used as the source for VH, Vκ-Cκ and Vλ-Cλ gene amplification via RT-PCR and nested PCR (FIGS. 2B, C). VH, Vκ-Cκ and Vλ-Cλ genes were sub cloned into the pTT5 mammalian expression vector and the sequences analysed (data not shown). Corresponding heavy and light chains originating from the same hit well were co-transfected into Expi293F cells for small scale whole IgG1 expression. From these co-transfections, recombinant mAbs that demonstrated binding to the original target were selected for large scale recombinant expression. These were then purified via affinity-based FPLC using a protein A resin and quality control checked via analytical mass spectrometry, SDS-PAGE gel analysis and analytical SEC (FIGS. 2D-G).

The IgG1 mAbs generated using the single B cell technology described above bound to purified Hyr1 protein (Table S3).

### Comparative Example 2

### Purified Recombinant Anti-Candida mAbs Exhibit Specific Target Binding

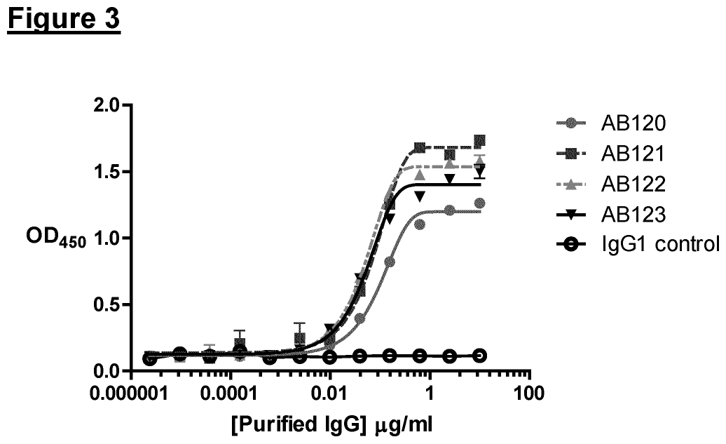

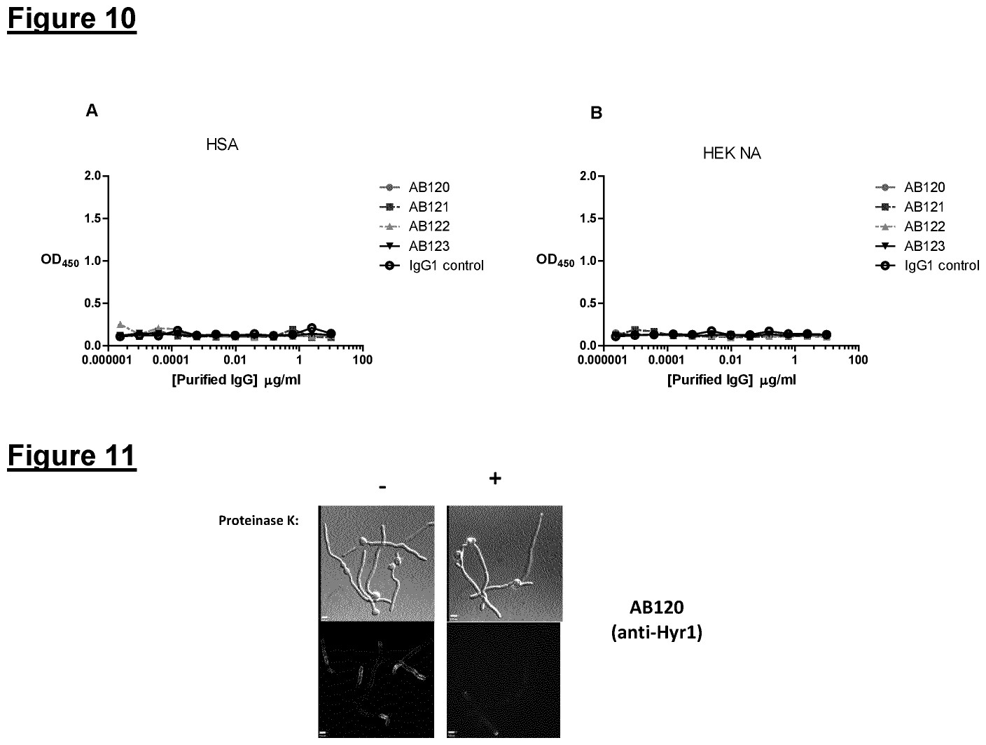

Purified anti-Hyr1 mAbs were primarily assessed for functionality through binding to the purified recombinant N-terminus of Hyr1 protein via ELISA. Four mAbs demonstrated strong binding to the purified antigen with EC₅₀ values of 104 ng/ml, 76.5 ng/ml, 49.6 ng/ml and 53.3 ng/ml for AB120, AB121, AB122 and AB123 (FIG. 3) respectively. To examine the specificity of these mAbs for the target protein, all four were tested against the unrelated antigens HSA and HEK nuclear antigen as negative controls and demonstrated no binding (FIG. 10).

### Comparative Example 3

### Purified Recombinant Anti-Candida mAbs Show Distinct Binding Patterns to C. albicans and Other Fungal Species

The recombinant anti-Hyr1 mAbs generated by single B cell technology were initially isolated by screening against N-terminus of Hyr1 protein and, following purification, demonstrated binding to this recombinant antigen (above). We then visualized binding of these mAbs to Hyr1 protein expressed on the C. albicans cell surface by immunofluorescent staining using a fluorescently labelled secondary anti-human IgG mAb for detection. It was observed that the anti-Hyr1 mAbs bound to the predicted cellular location on the hyphae, and not the WT C. albicans yeast cells grown in different culture conditions (FIG. 8A). We verified that the anti-Hyr1 mAbs did not bind to hyphae of a Δhyr1 null mutant (FIG. 8B) and that binding was restored in a C. albicans strain containing a single reintegrated copy of the deleted HYR1 gene (FIG. 8C).

C. albicans cells were enzymatically treated with proteinase K, endoglycosidase H (endo-H) and zymolyase 20T and assessed for mAb binding. Proteinase K treatment reduced AB120 (anti-Hyr1) (FIG. 11).

Commensurate with the C. albicans-specific nature of HYR1, anti-Hyr1 mAbs only bound to C. albicans and not to a range of other Candida species (results not shown).

In conclusion, all purified recombinant mAbs generated by this single B cell technology bound specifically to their target antigens with high affinity.

### Example 4

### Anti-Candida Hyr1 mAbs Bind Selectively to the Gram Negative Bacteria, Acinetobacter baumannii

Purified anti-Hyr1 mAbs were assessed for binding to a panel of Gram negative bacteria via ELISA. All four mAbs demonstrated strong binding to A. baumannii whole cells with EC₅₀ values of 6.4 ng/ml, 5.8 ng/ml, 4.3 ng/ml and 11.7 ng/ml for AB120, AB121, AB122 and AB123 respectively (FIG. 4 A-D). This binding was specific to A. baumannii and no cross reactivity to other Gram negative bacteria tested (E. coli, S. marcescens, and P. aueruginosa) was observed (FIG. 4 A-D). The IgG1 negative control mAb did not bind to any of the Gram negative bacteria tested (FIG. 4E).

### Example 5

### Indirect Immunofluorescence and TEM Images of Anti-CaHyr1 mAbs Binding to A. baumannii Cells

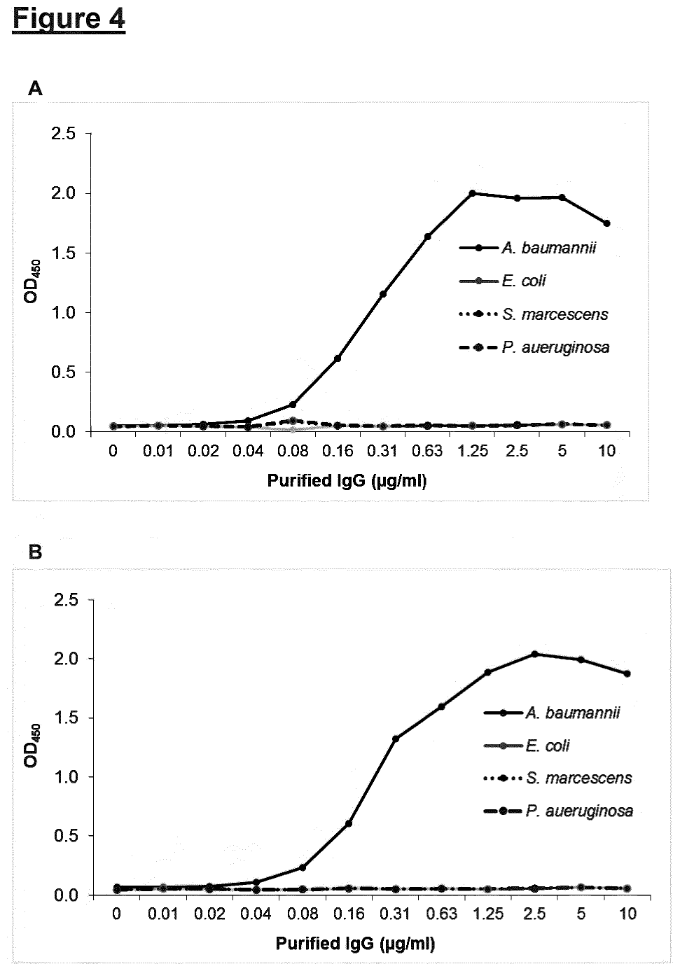

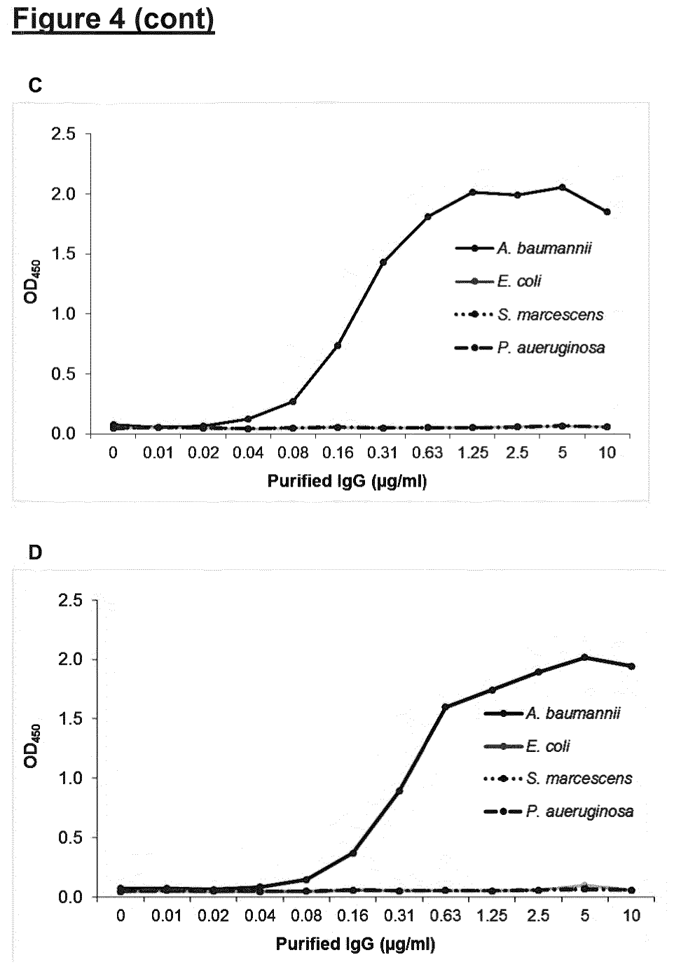

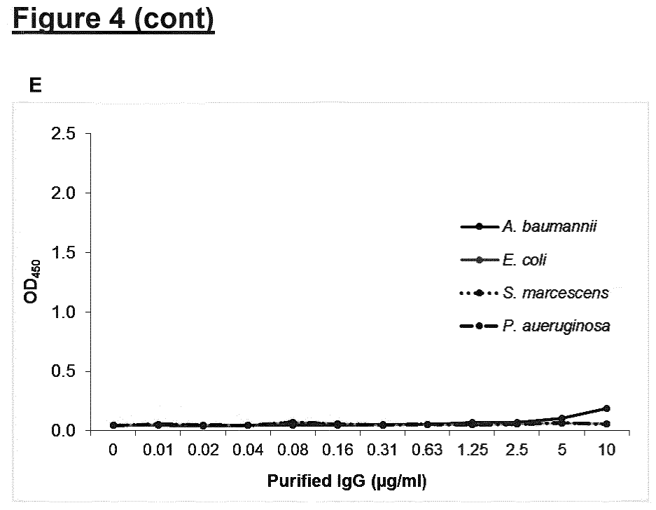

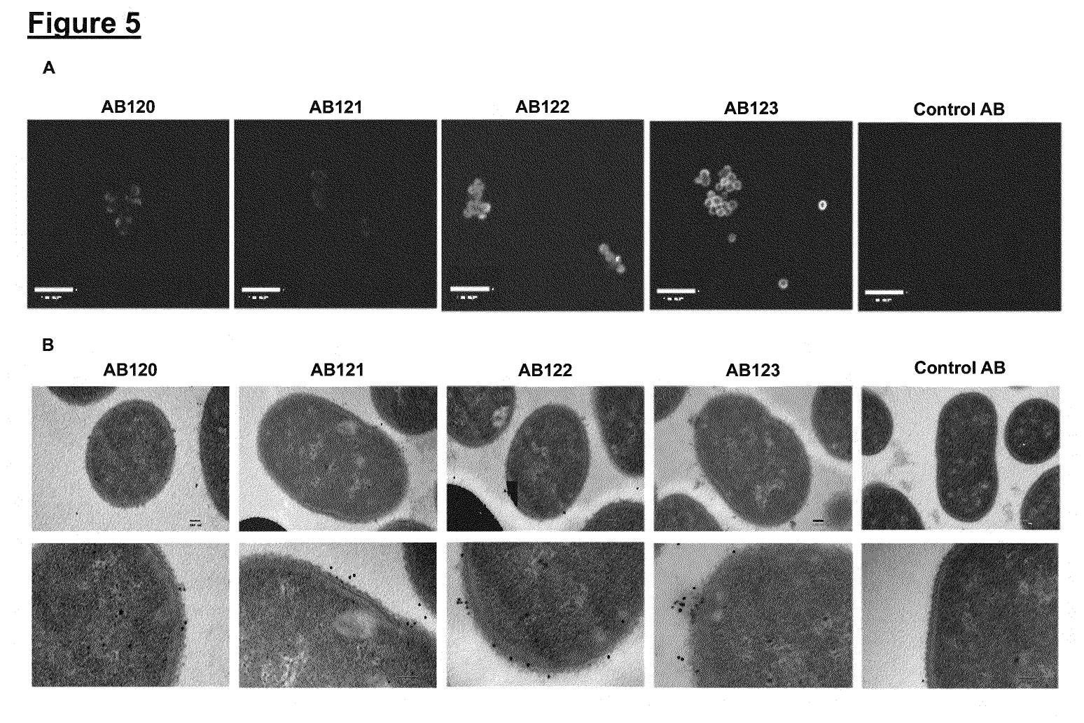

Binding of the four mAbs AB120-AB123 to A. baumannii was visualised by immunofluorescent staining using a fluorescently labelled alexa-488 conjugated secondary goat anti-human IgG antibody for detection. It was observed that all four anti-Hyr1 mAbs demonstrated binding to a target that was abundantly expressed on the A. baumannii cell surface (FIG. 5A). Transmission electron microscopy (TEM) revealed the ultrastructural binding of AB120-AB123 to the A. baumannii cell surface (FIG. 5B).

### Example 6

### Treatment with Anti-Hyr1 mAbs Promote Phagocytosis by Macrophages and Protects Against A. baumannii Infection

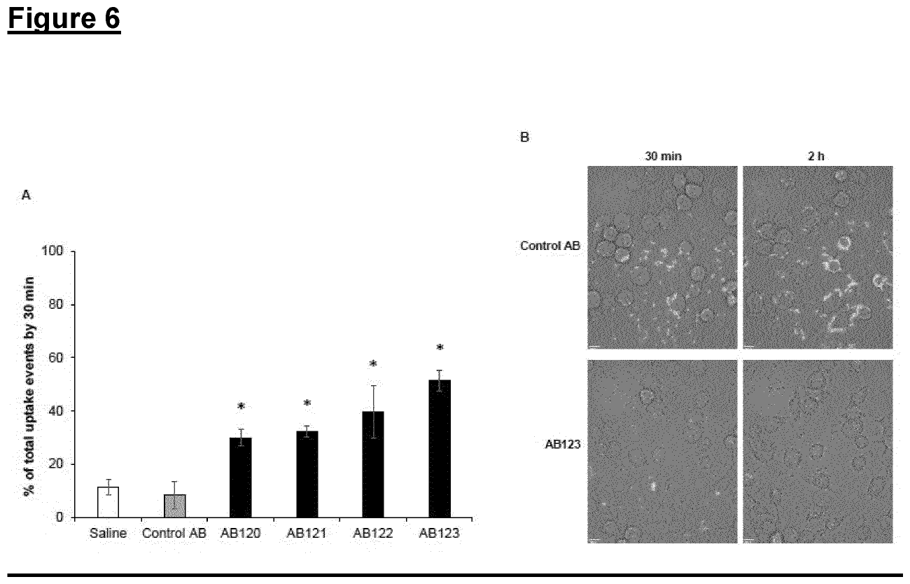

Phagocytic cells of the innate immune system are the first line of defence against microbial pathogens. Antibody binding enhances phagocytic clearance of pathogens. A live cell phagocytosis assay was utilised to examine whether the anti-Hyr1 mAbs generated in this study opsonized A. baumannii for phagocytosis by J774.1 macrophages. A. baumannii cells that had been pre-incubated with either AB120, AB121, AB122 or AB123 were taken up significantly more rapidly than cells which had been pre-incubated with saline or IgG1 control mAb. This was demonstrated through analysing the percentage of uptake events that had occurred within the first 30 min of the assay (FIG. 6A). Snapshots taken from live cell imaging compare the phagocytosis of A. baumannii cells by macrophages at 30 min and 2 h when bacterial cells had been pre-incubated with either 50 μg/ml control mAb or 50 μg/ml anti-Hyr1 mAb AB123 (FIG. 6B).

### Example 7

### Treatment with Anti-Hyr1 mAbs Protects Against A. baumannii Infection

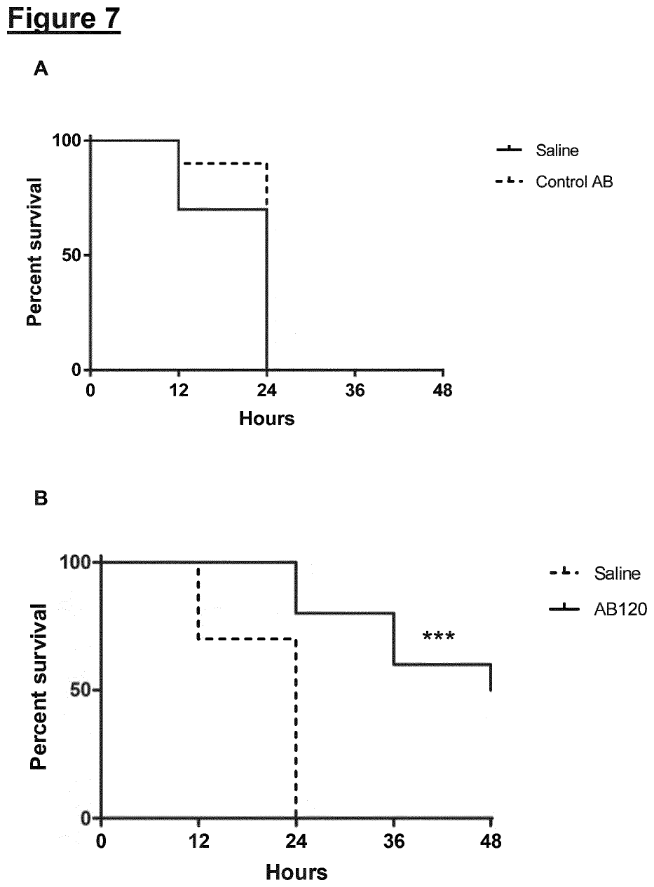

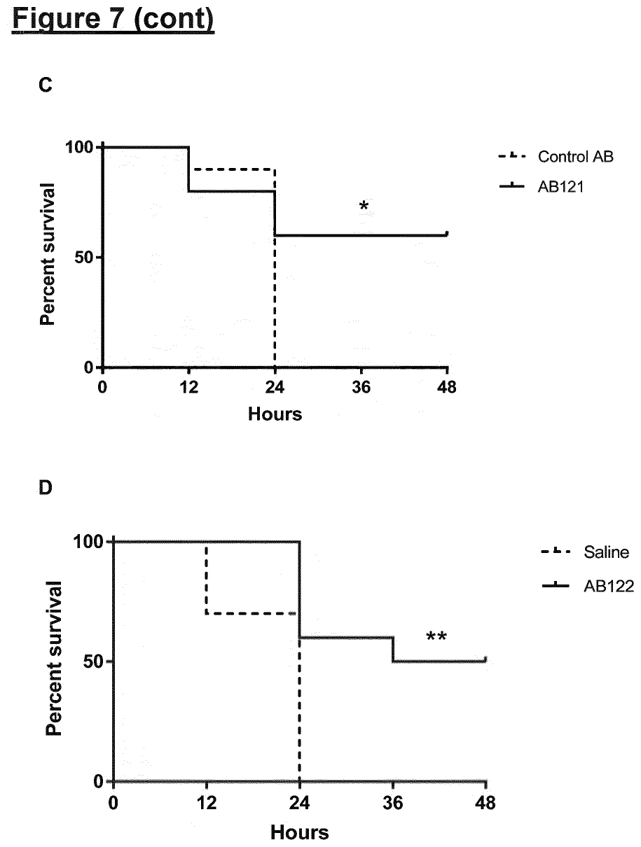

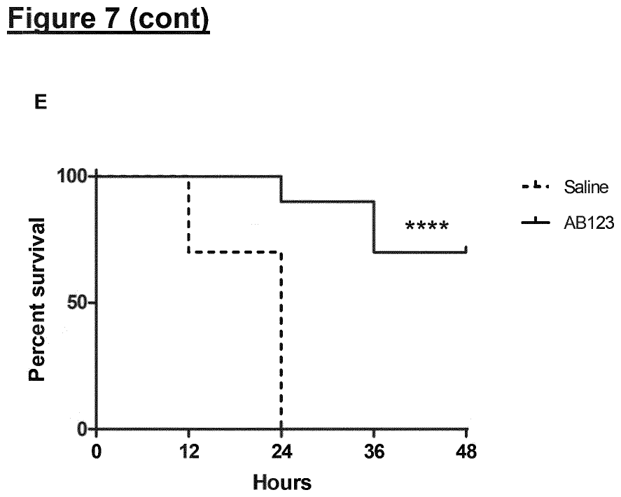

To determine the therapeutic potential of the anti-Hyr1 mAbs for treating A. baumannii infection, their activity was assessed in a Galleria mellonella model of systemic infection (FIG. 7). Galleria mellonella were infected with 1×10⁶ cells of A. baumannii following pre-incubation with saline (A) or 50 μg/ml IgG1 control mAb or anti-Hyr1 mAb AB120 (B), AB121 (C), AB122 (D), or AB123 (E), and their survival monitored every 12 h. Galleria mellonella in the saline and IgG1 control groups did not survive more than 24 hours whereas anti-Hyr1 mAb-treated groups survived significantly longer (p<0.005, p<0.05, p<0.01 and p<0.0001 for AB120, AB121, AB122 and AB123 vs IgG1 control, respectively). Galleria mellonella incubated with AB123 displayed the greatest survival, with around 60% surviving for up to 48 hours (p<0.0001 vs IgG1 control).

### Example 8

### Prevention of Biofilms

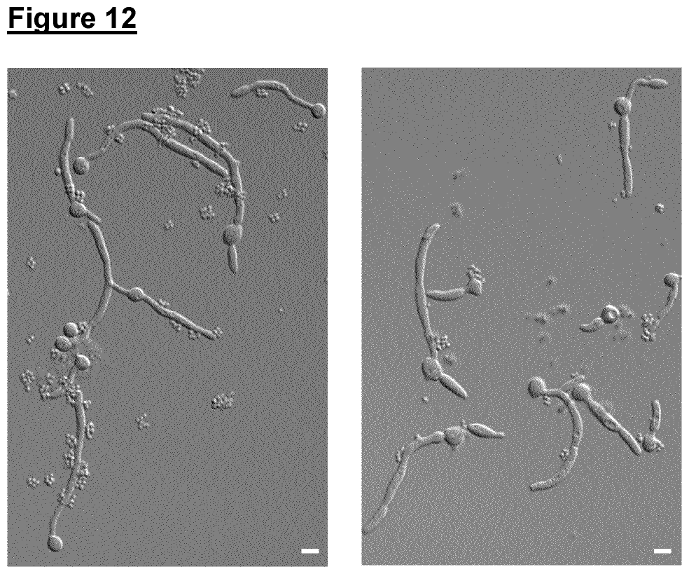

FIG. 12 demonstrates that treatment with mAb AB123 prevents biofilm formation between C. albicans and A. baumannii.

### Example 9

### Binding to Diverse Isolates

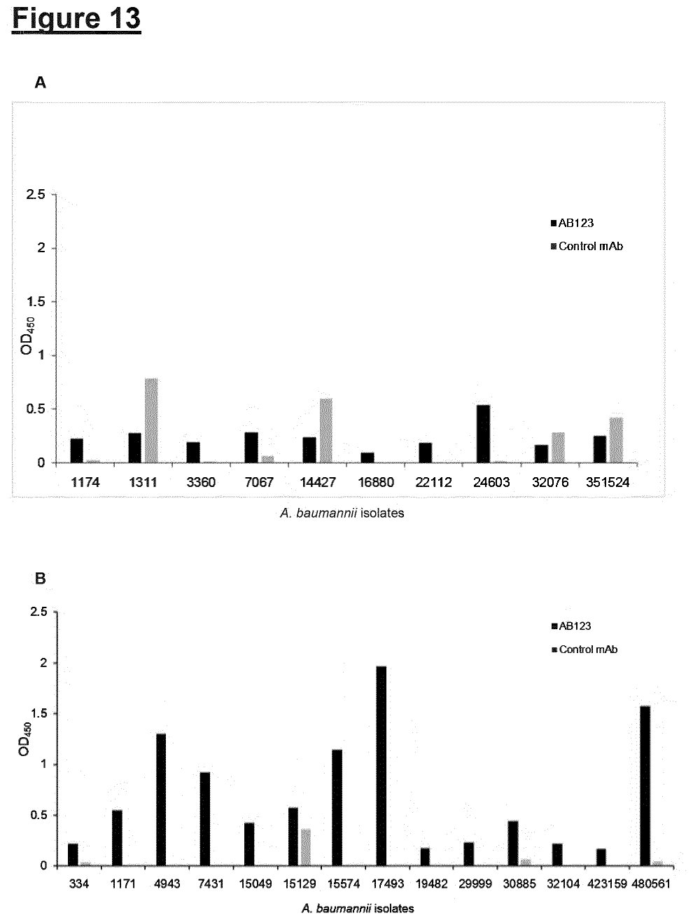

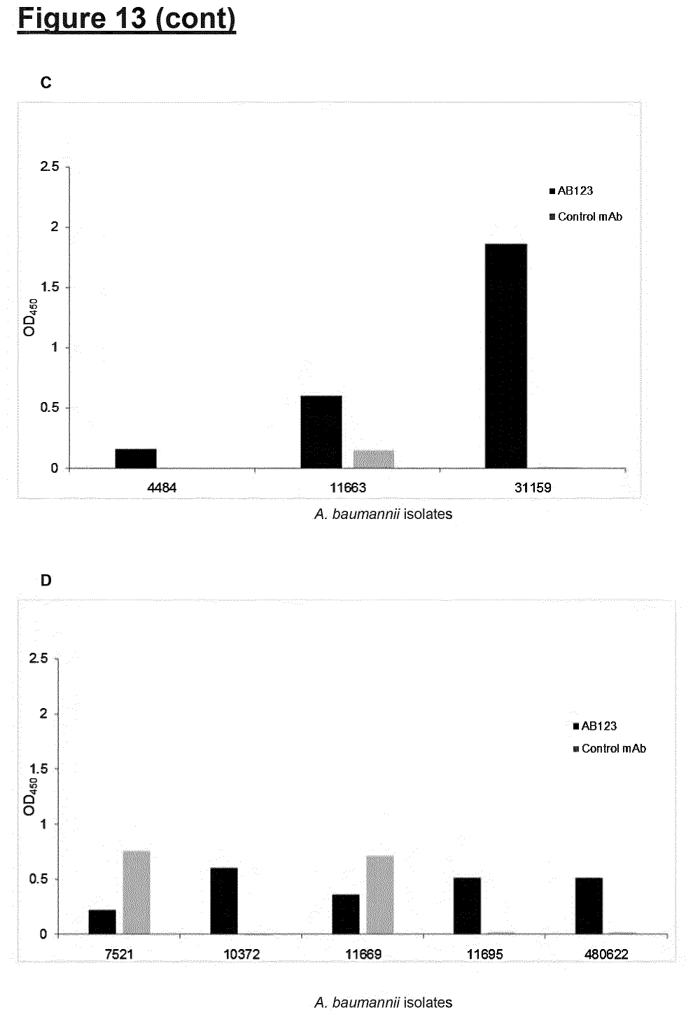

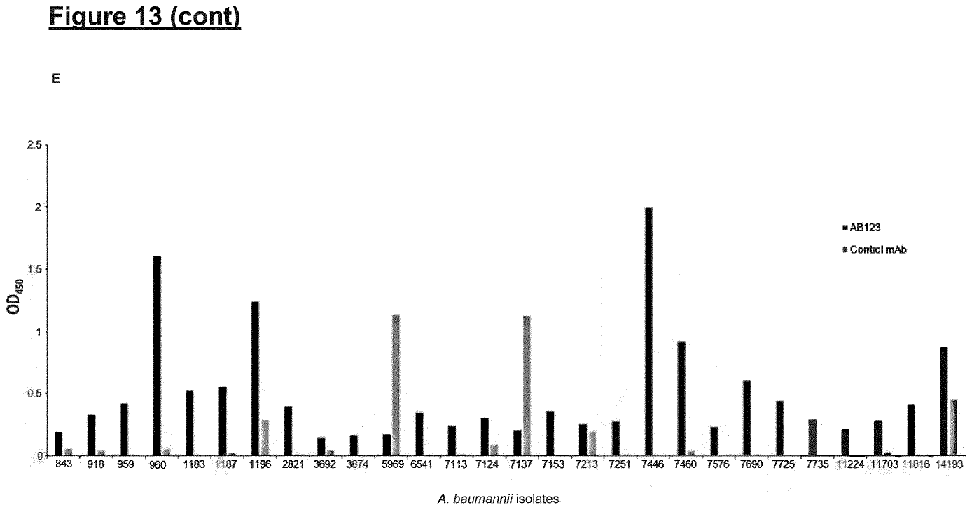

FIG. 13 is an expanded ELISA screen demonstrating mAb AB123 binding to a panel of diverse A. baumannii isolates isolated from different body sites.

General Methods

Candida and Gram Negative Bacteria Strains and Growth Conditions

C. albicans serotype A strain CA14+Clp10 (NGY152) was used as a control and its parent strain CA14, used to construct the Δhyr1 null mutant C. albicans strain (40) and the hyr1 re-integrant strain (unpublished). The clinical isolates C. albicans SC5314, C. glabrata SC571182B, C. tropicalis AM2005/0546, C. parapsilosis ATCC22019, C. lusitaniae SC5211362H, C. krusei SC571987M, C. dubliniensis CD36 are shown in Table 51. All strains were obtained from glycerol stocks stored at −80° C. and plated onto YPD plates (2% (w/v) mycological peptone (Oxoid, Cambridge, UK), 1% (w/v) yeast extract (Oxoid), 2% (w/v) glucose (Fisher Scientific, Leicestershire, UK) and 2% (w/v) technical agar (Oxoid)). Candida strains tested were routinely grown in YPD (see above without the technical agar). Acinetobacter baumannii strain ATCC 19606 is shown in Table 51. It was obtained from glycerol stocks stored at −80° C. and plated onto Mueller-Hinton agar and routinely grown in Mueller-Hinton broth. Isolates of Pseudomonas aeruginosa ATCC27853, Serratia marcescensDb10 and Escherichia coli AM2002/0068 (Table 51) were all obtained from −80° C. glycerol stocks and maintained on LB agar (1% (w/v) tryptone (Oxoid), 1% (w/v) NaCl (Fisher), 0.5% (w/v) yeast extract and 2% (w/v) technical agar) and routinely grown in LB broth at 37° C.

Generation of Recombinant Hyr1 N-Protein

The recombinant N-terminus of the Hyr1 protein (amino acids 63 to 350—Table S2) incorporating an N-terminal 6×xHis tag was expressed in HEK293F cells and purified by nickel-based affinity chromatography using a nickel NTA superflow column (QIAGEN, USA). Fractions containing the recombinant N-terminus of the Hyr1 protein were pooled and further purified via Analytical Superdex 200 gel filtration chromatography (GE Healthcare, USA) in PBS. QC of the recombinant protein via SDS-PAGE gel analysis, analytical size exclusion chromatography (SEC) and Western blot (using an anti-His antibody for detection) confirmed a protein of 32 kDa (data not shown).

Identification of Human Anti-Hyr1 mAbs from Donor B Cells PBMC Isolation

In brief, peripheral venous blood from donors who had recovered from a Candida infection within the last year was collected in EDTA-coated vacutainers tubes and pooled. PBMCs and plasma were separated from the whole blood suspension via density gradient separation using Accuspin System-Histopaque-1077 kits (Sigma-Aldrich) according to manufacturer's instructions. Following separation, the plasma layer was aspirated and stored at 4° C. for later analysis of antibody titre and the PBMC layer was aspirated and washed in PBS and centrifugation at 250×g for 10 min three times before final resuspension at a concentration of 1×10⁷ cells/ml in R10 media (RPMI 1640 (Gibco, Life Technologies), 10% FCS, 1 mM sodium pyruvate (Sigma), 10 mM HEPES (Gibco, Life Technologies), 4 mM L-glutamine (Sigma), 1× penicillin/streptomycin (Sigma)) containing additional 10% FCS and 10% DMSO. PBMCs were split into 1 ml aliquots and stored in liquid nitrogen until they were required.

Purification of Donor Plasma

IgG was purified from donor plasma using VivaPure MaxiPrepG Spin columns (Sartorius Stedman) according to manufacturer's instructions. In brief, plasma sample was applied to the spin column to facilitate IgG binding. The column was washed twice in PBS and then bound IgG was eluted in an amine buffer, pH 2.5 and neutralized with 1 M Tris buffer, pH 8. Eluted IgG concentration was measured by absorbance at 280 nm using a NanoVue Plus Spectrophotometer (GE Healthcare).

Circulating IgG Enzyme-linked Immunosorbent Assay (ELISA) to Identify Donors with B Cells to Take Forward

To identify the donor to use for subsequent class switched memory (CSM) B cell isolation and activation, ELISAs were carried out against the target antigen using IgG purified from donor plasma. NUNC maxisorp 384-well plates (Sigma) were coated with 1 μg/ml purified, recombinant N-terminus hyrl protein antigen in 1×PBS and incubated at 4° C. overnight. The next day, wells were washed three times with wash buffer (1×PBS+0.05% Tween) using a Zoom Microplate Washer (Titertek). Wells were then blocked with block buffer (1×PBS+0.05% Tween+0.5% BSA) for 1 h at room temperature with gentle shaking to inhibit non-specific binding. After three washes (as above), titrated purified IgG or IVIG in block buffer was added in duplicate, and the plates were incubated for 2 h at room temperature with gentle shaking. Wells were washed with wash buffer as above before addition of goat anti-human IgG, HRP conjugated (ThermoScientific) secondary antibody at 1:5000 dilution in blocking buffer. Plates were incubated for 45 min at room temperature with gentle shaking.

To develop the ELISA, wells were washed three times with wash buffer (as above) before the addition of TMB (Thermo Scientific). Plates were incubated at room temperature for 5 min to allow the blue colour to develop and the reaction was quenched by the addition of 0.18 M sulphuric acid. The plates were then read at an OD of 450 nm on an Envision plate reader (PerkinElmer). Labstats software in Microsoft Excel was used to generate concentration-response curves for EC₅₀ determination and donor selection for subsequent CSM B cell isolation and activation.

Isolation of Class Switched Memory B Cells

The PBMCs from donors who displayed a strong IgG response to the antigen of interest in the screening ELISA were taken forward for CSM B cell isolation and activation. The process of generating recombinant mAbs from a single donor's B cells to one particular antigen, beginning with the isolation of CSM B cells all the way through to expression and purification of recombinant mAbs, was termed an ‘Activation’. For each Activation, 5×10⁷ PBMCs were removed from the liquid nitrogen store and thawed by adding pre-warmed R10 media drop wise to the cells. The diluted cell suspension was then transferred into a fresh polypropylene tube containing pre-warmed R10, resulting in a final cell dilution of approximately 1:10. Benzonase nuclease HC, purity >99% (Novagen) was added at a 1:10000 dilution (to ensure any lysed cells and their components didn't interfere with the live cells), and the cells were centrifuged at 300×g for 10 min at room temperature and the supernatant removed. PBMCs were then washed again in R10 before final resuspension in 1 ml R10 for PBMC cell number and viability determination.

Isolation of class switched memory B cells from PBMCs was carried out by magnetic bead separation using a Switched Memory B cell isolation kit with Pre-Separation Filters and LS columns (MACS Miltenyi Biotec) according to manufacturer's instructions. In brief, counted PBMCs were incubated with a cocktail of biotin-conjugated antibodies against CD2, CD14, CD16, CD36, CD43, CD235a (glycophorin A), IgM and IgD. Cells were then washed and incubated with anti-biotin microbeads. Following another wash step, the suspension was passed through a Pre-Separation Filter (to remove cell aggregates) before applying it to an LS column where the magnetically labelled cells were retained in the column and the unlabelled CSM B cells passed through and could be collected in the flow-through for determination of cell number and viability.

Activation of CSM B Cells

To activate CSM B cells and promote antibody secretion into the supernatant, a mixture of cytokines, mAb, TLR agonist and a supplement were added to the R10 media (see above) to make complete R10 media. CSM B cells were resuspended in complete R10 media at 56 cells/ml and then plated out at 90 μl/well (5 cells/well) in ThermoFisher Matrix 384 well plates using a Biomek FX (Beckman Coulter). Cells were incubated at 37° C., 5% CO₂ for seven days. On day 7, 30 μl/well of supernatant was removed and replaced with 30 μl fresh complete R10. On day 13, all the supernatant was harvested from all plates and screened against the antigen of interest via ELISA. B cell activation and culturing was monitored by measuring IgG1 concentrations in B cell supernatants at day 7 and day 13.

B Cell Supernatant Screen Against Target Antigens Via ELISA

For B cell supernatant screening against target antigens, NUNC maxisorp 384-well plates (Sigma) were coated with 1 μg/ml purified, recombinant N-terminus hyr1 protein antigen in 1×PBS and incubated at 4° C. overnight. Wells were washed three times with wash buffer using a Zoom Microplate Washer (Titertek) as above before incubation with blocking buffer for 1 h at room temperature with gentle shaking. After another three washes (as above), B cell supernatant was added and the plates incubated for 2 h at room temperature with gentle shaking. Wells were washed with wash buffer as above before addition of goat anti-human IgG, HRP conjugated (ThermoScientific) secondary antibody at 1:5000 dilution in blocking buffer and incubation for 45 min at room temperature with gentle shaking. ELISAs were developed and plates read at an OD of 450nm on an Envision plate reader (PerkinElmer). Positive hits were defined as wells with an OD₄₅₀ reading>4×background. B cells in ‘positive hit’ wells were resuspended in lysis buffer (ml DEPC-treated H₂O (Life Technologies), 10 μl 1 M Tris pH 8, 25 μl RNAsin Plus RNAse Inhibitor (Promega)) and stored at −80° C.

Generation of Recombinant Anti-Hyr1 mAbs: Amplification of VH, Vκ-Cκ and Vλ-Cλ Genes—cDNA Synthesis and PCR

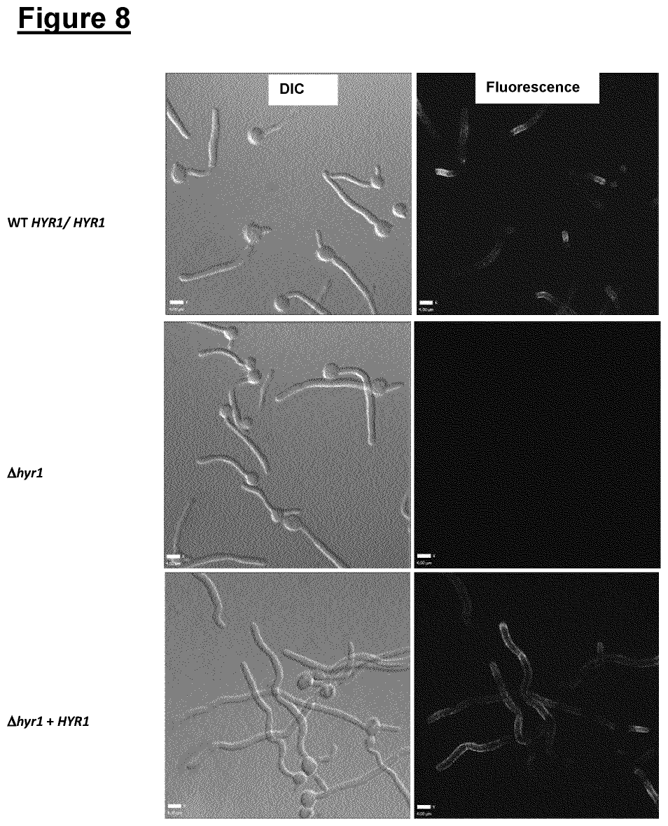

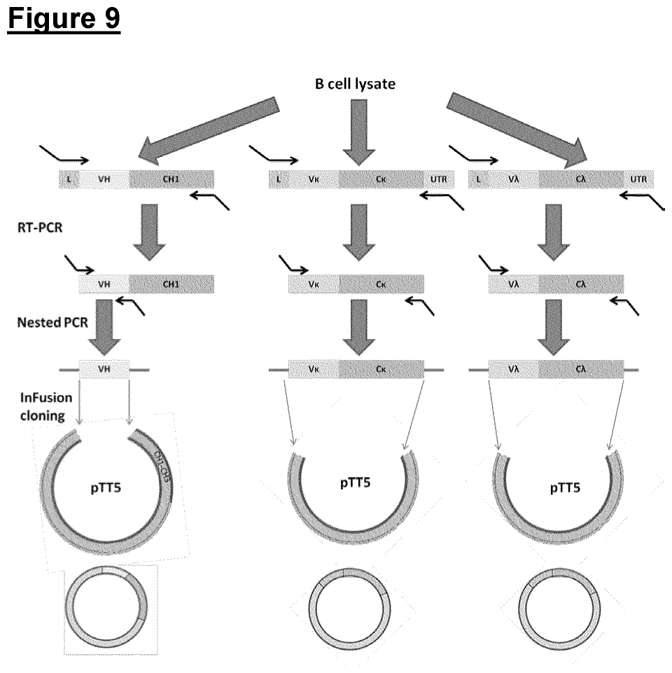

A schematic of the cloning protocol is shown in FIG. 9. Primers used for the RT-PCR reaction were based on those used by Smith et. al., (36). To ensure all possible VH germline families were captured during the amplification, four forward primers specific to the leader sequences encompassing the different human VH germline families (VH1-7) were used in combination with two reverse primers; both placed in the human CgCH1 region. For the RT-PCR of human Vκ-Cκ genes, three forward primers specific to the leader sequences for the different human Vκ germline families (VK1-4) were used with a reverse primer specific to the human kappa constant region (CK) and two further reverse primers which were specific to the C- and N-terminal ends of the 3′ untranslated region (UTR). To capture the repertoire of human Vλ genes, 7 forward primers capturing the leader sequences for the different human Vλ germline families (VA1-8) were used in a mixture with two reverse primers which were complementary to the C- and N-terminal ends of the 3′ UTR and another reverse primer specific to the human lambda constant region (CA).

Prior to cDNA synthesis, B cell lysates were thawed and diluted 1:5, 1:15 and 1:25 in nuclease-free H₂O (Life Technologies) before addition of oligodT₂₀ (50 μM) (Invitrogen, Life Technologies) and incubation at 70° C. for 5 min. Reverse transcription and the first PCR reaction (RT-PCR) were done sequentially using the QIAGEN OneStep RT-PCR kit according to manufacturer's instructions. For this step and the subsequent nested PCR step, amplification of the variable domain of human Ig heavy chain genes (VH), the variable and constant domains of human Ig kappa light chain genes (Vθ-Cκ) and the variable and constant domains of human Ig lambda light chain genes (Vλ-Cλ), were done in separate reactions. In brief, a reaction mixture was prepared containing QIAGEN OneStep RT-PCR Buffer 5×, dNTPs (10 mM), gene-specific forward and reverse primer mixes (10 μM), QIAGEN OneStep RT-PCR Enzyme Mix and nuclease-free H₂O. Reaction mixture was then added to wells of a 96-well PCR plate before addition of neat or diluted (1:5, 1:15, 1:25) B cell lysate as the template, resulting in a final reaction volume of 50 μl/well. The following cycling conditions were used for the RT-PCR reaction; 50° C. for 30 min, 95° C. for 15 min then 35-40 cycles of (94° C. for 1 min, 55° C. for 1 min and 72° C. for 1 min) with a final extension at 72° C. for 10 min.

Amplification of VH, Vκ-Cκ and Vλ-Cλ Genes—Nested PCR Reaction

Nested PCR reactions were carried out using the PCR products from the RT-PCR reaction as the template, nested gene-specific primers based on Smith et al. (36) and Platinum PCR SuperMix High-Fidelity (Invitrogen, Life Technologies). A total of 27 forward primers specific for the VH framework 1 (FW1) sequence were used together with two reverse primers specific for the framework 4 (FW4) region of the VH gene. For nested PCR of the Vκ-Cκ gene, a mixture of 18 forward primers specific for human Vκ FW1 sequence were used with a reverse primer specific to the human kappa constant region 3′ end. For amplification of the Vλ-Cλ gene, a mixture of 31 forward primers specific for human Vλ FW1 sequences were used together with a reverse primer that was placed at the 3′ end of the human lambda constant region. The primers used to generate the PCR fragments in these nested PCR reactions contained 15 bp extensions which were complementary to the target downstream pTT5 expression vector. Reaction mixtures containing Platinum PCR SuperMix High Fidelity, gene-specific forward primer mix (10 μM) and gene specific reverse primer mix (10 μM) was added to wells in a 96-well PCR plate before addition of cDNA template. Amplification of VH genes, Vκ-Cκ genes and Vλ-Cλ genes, were done in separate reactions. After the nested PCR reaction, samples were analysed via agarose gel electrophoresis and positive hits identified and taken forward for downstream InFusion cloning with pTT5 mammalian expression vector.

pTT5 Mammalian Expression Vector Preparation

The pTT5mammalian expression used for mAb expression (licensed from the National Research Council of Canada (NRCC)) (53). The pTT5 vector plasmid contained an IgG1 heavy chain gene in the multiple cloning site so digestion to generate the heavy chain (HC) backbone for downstream sub cloning of VH was done by double digestion using FastDigest Restriction enzymes (Thermo Scientific) with BssHll before the leader sequence of the VH region and Sall restriction after the FW4 of the VH domain. This yielded the heavy chain constant region in the vector backbone. For double digestion of the vector to generate the light chain (LC) backbone, the whole IgG1 heavy chain gene was with BssHll and BamHl astDigest Restriction enzymes (Thermo Scientific) to generate the vector ready for insertion of either K-Cκ or VA-CA. Digestion reactions to generate HC and LC backbones were carried out separately. Following confirmation of digestion, samples were run on a 1% agarose gel and bands were excised from the gel and purified using the QlAquick Gel Extraction kit (QIAGEN). DNA was quantified on a NanoVue Plus Spectrophotometer (GE Healthcare). To prevent vector self-ligation, the 3′- and 5′-termini of the linearized plasmids were dephosphorylated using FastAP Thermosensitive Alkaline phosphatase (Thermo Scientific). Reaction mixtures were cleaned up using the MinElute Reaction Cleanup Kit (QIAGEN) and then run on a 1% agarose gel. Bands corresponding to dephosphorylated HC and LC backbones were excised from the gel and purified using the QIAQuick Gel Extraction kit (QIAGEN) as above. Dephosphorylated linearized vector DNA was quantified on a NanoVue Plus spectrophotometer (GE Healthcare).

In-Fusion Cloning

The In-Fusion HD Cloning Kit (Clontech, USA) was used to clone the IgG VH, Vκ-Cκ and Vλ-Cλ genes into a pTT5 mammalian expression vector. To avoid the need for nested PCR product purification before cloning, cloning enhancer (Clontech, USA) was added to each nested PCR product in a 96-well PCR plate and incubated at 37° C. for 15 min, then 80° C. for 15 min. The cloning enhancer-treated PCR product was then added to the In-Fusion Enzyme Premix and linearized vector DNA (˜-5-10 ng). Reactions were made up to 10 μl with nuclease-free H₂O and incubated for 15 min at 50° C. Samples were then either stored at -20° C. or placed on ice before transformation of Stellar Competent cells (Clontech). For transformation, 2 μl of each In-Fusion reaction mixture was added to cells in a 96-well plate format, and left on ice for 30 min before heat shock at 42° C. for 40 sec and then returning to ice for 2 min. Cells were then recovered in SOC medium (Clontech, USA) with gentle shaking at 37° C. for 45-60 min before plating out onto LB agar plates (1% (w/v) tryptone, 0.5% (w/v) yeast extract, 1% (w/v) NaCl, 1.5% (w/v) agar) containing 100 μg/ml ampicillin. Plates were incubated at 37° C. overnight and single colonies picked the next day.

Plasmid DNA Generation for Transfection

Following transformation, 8-16 single colonies per initial hit well for VH, Vκ and Vλ were picked and used to inoculate 2xTY media containing 100 μg/ml ampicillin in a Greiner deep well, 96-well plate (Sigma). VH, Vκ and Vλ plates were set up separately with the same plate layout to facilitate visual screening. Cells were grown at 37° C., 200 rpm overnight, and glycerol stocks were made the following day and stored at −80° C. To ensure accurate tracking of DNA sequences for downstream sequencing and transfections, each well inoculated by a single colony was given a unique ID based on the colony's original hit well and its position in the deep well 96 well plate following transformations. To obtain plasmid DNA for gene sequencing and small scale mammalian transfections, DNA minipreps from the overnight cultures were carried out in a 96-well plate format using the EPmotion (Eppendorf), according to manufacturer's instructions. DNA not taken for gene sequencing was stored at −20° C. until required for small scale transfections. Sequence data was analysed for CDR diversity and comparisons to germline sequences and used to identify clones to take forward for small scale transfection.

Small Scale Expression of Recombinant mAbs

Following VH, Vκ and Vλ gene sequencing, a file was generated containing all possible VH and Vκ/Vλ combinations resulting from the original hit wells from the primary ELISA screen. Automated mixing of the native heavy and light chain DNA pairing combinations (1.5 μg of HC plasmid DNA and 1.5 μg of LC plasmid DNA) into a new 96-well plate was facilitated through a HAMILTON MICROLAB® Starline liquid handling platform (Life Science robotics, Hamilton Robotics). Subsequent mixed DNA was used for small scale transient transfection of 3 ml of suspension cultured Expi293F cells (Life Technologies, USA) at a density of 2.5×10⁶ cells/ml in 24-well tissue culture plates using the Expifectamine 293 Transfection kit (Life Technologies, USA) in accordance with manufacturer's instructions. Expi293F cells were maintained in pre-warmed (37° C.) sterile Expi293 expression media (Invitrogen) without antibiotics at 37° C., 7% CO₂, 120 rpm shaking. Supernatants were harvested on day 6 and recombinant mAb expression was quantified using anti-human IgG Fc sensors on an Octet QKe (ForteBio, CA, USA) for identification of mAbs to upscale.

Large Scale Expression, Purification and QC of Recombinant mAbs

For downstream large scale mammalian transfections, where a greater amount of DNA was required, DNA was prepared using a QIAGEN Plasmid Maxi Kit (QIAGEN, USA) according to manufacturer's instructions with typical yields of 1.5 μg/μl. For large scale mAb expression, 100 μg of total DNA (50 μg of HC plasmid DNA and 50 μg LC plasmid DNA) was used to transiently transfect 100 ml of suspension cultured Expi293F cells (Life Technologies, USA) at a density of 2.5×10⁶ cells/ml using the Expifectamine 293 Transfection Kit (Life Technologies, USA) in accordance with the manufacturer's instructions. Supernatants were harvested on day 6 and recombinant mAb expression was quantified as above using an Octet QKe (ForteBio). Recombinant mAbs were purified via affinity based Fast Protein Liquid Chromatography using HiTrap Protein A HP columns on an AKTA (GE Healthcare) and eluted in 20 mM citric acid, 150 nM NaCl (pH2.5) before neutralisation with 1 M Tris buffer (pH8). Purified mAbs were dialysed in PBS overnight and IgG concentration was quantified on a NanoVue Spectrophotometer (GE Healthcare). All purified recombinant mAbs were quality control checked via SDS-PAGE gel analysis using 4-12% Bis-Tris SDS-PAGE gels under reducing and non-reducing conditions to confirm mass, analytical size exclusion chromatography (SEC) to check for protein aggregation/degradation and analytical mass spectrometry to confirm the amino acid sequence identity of each mAb. Purified recombinant mAbs were also tested for functionality by binding to target antigen/whole cell via ELISA.

ELISA with Purified Recombinant mAbs

For confirmation of binding to target as purified recombinant mAbs an ELISA was carried out using the protocol for B cell supernatant screen. The only change was that titrated purified recombinant mAb was added in place of B cell supernatant. For assessment of mAb binding to A. baumannii, A. baumannii overnight culture was coated onto plates in place of 1 μg/ml purified, recombinant N-terminus hyrl protein antigen.

Immunofluorescence Imaging of Anti-Hyr1 mAbs Binding to Fungal Cells

Indirect immunofluorescence was performed using purified recombinant mAbs. For A. baumannii staining, a single A. baumannii colony was used to inoculate 10 ml Mueller-Hinton medium and incubated at 37° C., 200 rpm overnight. For imaging of Candida cells, a single Candida colony was used to inoculate 10 ml YPD medium and incubated at 30° C., 200 rpm overnight. Overnight Candida cultures were diluted 1:1333 in milliQ water and then added to a poly-L-lysine coated glass slide (Thermo Scientific, Menzel-Glaser) and incubated for 30 min at room temperature to allow for adherence of yeast cells to the slide. To induce filamentation, cells were incubated in pre-warmed RPMI+10% FCS at 37° C. for 90 min-2 h (this step was omitted for staining of yeast cells). A. baumannii and Candida cells were then washed in Dulbecco's Phosphate Buffered Saline (DPBS) and fixed with 4% paraformaldehyde for 30 min. After fixing, cells were washed again and blocked with 1.5% normal goat serum (Life Technologies) before staining with an anti-Hyr1 mAb at 10 μg/ml for 1 h at room temperature. After three PBS washes, cells were stained with Alexa Fluor® 488 goat anti-human IgG antibody (Life Technologies) at a 1:400 dilution and incubated at room temperature for 1 h in the dark. For additional staining of fungal cell wall chitin in Candida cells, Calcofluor White (CFW) was added at 25 μg/ml and cells were incubated for 10 min at room temperature in the dark and washed with DPBS. Slides with Candida cells were left to air dry, A. baumannii cells were washed three time with DPBS and 5 μl of cell suspension added to a poly-L-lysine coated glass slide. One drop of Vectashield mounting medium (Vector Labs) was added to the slides and a 20 m×20 mm coverslip applied. Cells were imaged in 3D on an UltraVIEW® VoX spinning disk confocal microscope (Nikon, Surrey, UK).

High-Pressure Freezing (HPF) of Samples for Immunogold Labelling of A. baumannii Cells with anti-Hyr1 mAbs for Transmission Electron Microscopy (TEM)

A. baumannii cell samples were prepared by high-pressure freezing using an EMPACT2 high-pressure freezer and rapid transport system (Leica Microsystems Ltd., Milton Keynes, United Kingdom). The frozen samples were then fixed in an automatic temperature controlled Leica AFS freeze substitution system in dried acetone containing 2% (w/v) OsO₄, 1% (w/v) uranyl acetate, 1% (v/v) methanol and 5% (v/v) water in acetone at −90° C. for 48 h (Walther & Ziegler, 2002). Samples were then warmed to −30° C. and processed in a Lynx tissue processor with 1:2 acetone: resin and embedding in TAAB812 (TAAB Laboratories, Aldermaston, UK) epoxy resin. One hundred nm sections were cut with a Leica ultracut E and placed on nickel grids. Sections were blocked in blocking buffer (PBS+1% (w/v) BSA and 0.5% (v/v) Tween20) for 20 min before incubation in incubation buffer (PBS+0.1% (w/v) BSA) for 5 min×3. Sections were then incubated with anti-Candida mAb (5 μg/ml) for 90 min before incubation in incubation buffer for 5 min, for a total of 6 times. mAb binding was detected by incubation with Protein A conjugated to 10 nm gold (Aurion) (diluted 1:40 in incubation buffer) for 60 min before another six, 5 min washes, in incubation buffer, followed by three, 5 min washes in PBS and three, 5 min washes in water. Sections were then stained with uranyl acetate for 1 min before three, 2 min washes, in water and left to dry. TEM images were taken using a JEM-1400 Plus using an AMT UltraVUE camera.

Preparation of J774.1 mouse macrophage cell line J774.1 macrophages (ECACC, HPA, Salisbury, UK) were maintained in tissue culture flasks in DMEM medium (Lonza, Slough, UK) supplemented with 10% (v/v) FCS (Biosera, Ringmer, UK), 200 U/ml penicillin/streptomycin antibiotics (Invitrogen, Paisley, UK) and 2 mM L-glutamine (Invitrogen, Paisley, UK) and incubated at 37° C., 5% CO₂. For phagocytosis assays, macrophages were seeded in 300 μl supplemented DMEM at a density of 2×10⁵ cells/well in an 8-well glass based imaging dish (Ibidi, Munich, Germany) and incubated overnight at 37° C., 5% CO₂. Immediately prior to phagocytosis experiments, supplemented DMEM was replaced with 300 μl pre-warmed supplemented CO₂-independent media (Gibco, Invitrogen, Paisley, UK) containing 1 μM LysoTracker Red DND-99 (Invitrogen, Paisley, UK).

Preparation of Fluorescein Isothiocyanate (FITC)-Stained A. baumannii

A. baumannii colonies were grown in Mueller-Hinton medium and incubated at 370° C., 200 rpm overnight. Live A. baumannii cells were stained for 10 min at room temperature in the dark with 1 mg/ml FITC (Sigma, Dorset, UK) in 0.05 M carbonate-bicarbonate buffer (pH 9.6) (BDH Chemicals, VWR International, Leicestershire, UK). Following the 10 min incubation, in phagocytosis assays using A. baumannii FITC-labelled, the cells were washed three times in 1 x PBS to remove any residual FITC and finally re-suspended in 1×PBS or 1×PBS containing purified anti-Candida mAb at 1-50 μg/ml.

Live Cell Video Microscopy Phagocytosis Assays

Phagocytosis assays were performed using our standard protocol with modifications (42, 43, 54). Following pre-incubation with/without anti-Hyr1 mAb, live FITC-stained wild type A. baumannii cells were added to LysoTracker Red DND-99-stained J774.1 murine macrophages or human monocyte-derived macrophages in an 8-well glass based imaging dish (Ibidi) at a multiplicity of infection (MOI) of 10. Video microscopy was performed using an UltraVIEW® VoX spinning disk confocal microscope (Nikon, Surrey, UK) in a 37° C. chamber and images were captured at 1 min intervals over a 4 h period. At least three independent experiments were performed for each antibody and at least 2 videos were analysed from each experiment using Volocity 6.3 imaging analysis software (Improvision, PerkinElmer, Coventry, UK). Twenty five macrophages were selected at random from each experiment and analysed individually at 1 min intervals over a 4 h period. Measurements taken included: A. baumannii uptake—defined as the number of A. baumannii cells taken up by an individual phagocyte over the 4 h period.

Mean values and standard deviations were calculated. One- or two-way ANOVλ followed by Bonferroni multiple comparison tests or unpaired, two-tailed t tests were used to determine statistical significance.

Enzymatic Modification of Candida albicans Cell Wall

For proteinase K treatment, single colonies of Candida were inoculated into 10 ml YPD medium and incubated at 30° C., 200 rpm overnight. Cultures were diluted in milliQ water and then adhered on poly-L-lysine coated glass slides. To induce filamentation, cells were incubated in pre-warmed RPMI+10% FCS at 37° C. for 90 min-2 h. Slides were washed with DPBS and cells were treated with 50 μg/mI proteinase K at 37° C. for 1 h. Cells were then washed in DPBS and fixed with 4% paraformaldehyde, washed and blocked with 1.5% normal goat serum (Life Technologies) before staining with an anti-Hyr1 mAb at 1 μg/mI for 1 h at room temperature. After 3 washes with DPBS, cells were stained with Alexa Fluor® 488 goat anti-human IgG antibody (Life Technologies) at a 1:400 dilution and incubated at room temperature for 1 h prior to imaging in 3D on an UltraVIEW® VoX spinning disk confocal microscope (Nikon, Surrey, UK).

Galleria mellonella Infection with A. baumannii and Treatment with Anti-Hyr1 mAbs

Sixth instar larvae of Galleria mellonella (Livefoodsbypost, Isle of Wight, England) were separated into 10 healthy larvae per treatment group. Overnight cultures of A. baumannii were diluted 1 in 100 in either PBS or PBS with 10 μg/ml of anti-Hyr1 mAb for 1 h at room temperature. Bacterial cells were then standardised to an OD₆₀₀ of 0.5, which corresponded to 10⁶ bacterial cells/ml. Larvae were inoculated with 10 μl of each culture through the last left pro-leg and incubated at 37° C. Survival of larvae was recorded every 12 h.

Biofilm Assay

Biofilms were formed by diluting an overnight culture of C. albicans yeast cells in pre-warmed RPMI-1640 +10% FCS to a concentration of 1×10⁶ cells/ml. The C. albicans culture was added to a poly-L-lysine coated glass slide (Thermo Scientific, Menzel-Glaser) and incubated for 2 h at 37° C. to induce hyphal formation. After incubation cells were washed with Dulbecco's Phosphate Buffered Saline (DPBS) and treated with either 50 μg/ml of control IgG1 mAb or 50 μg/ml of AB123 for 1 h at RT. Cells were then washed three times with DPBS. Overnight cultures of A. baumannii were washed three times with DPBS and diluted to 1×10⁵ cells/ml in pre-warmed RPMI-1640 +10% FCS and added to the C. albicans hyphae. Cells were then co-incubated at 37° C. for 3 h. Cells were then washed three times with DPBS and fixed with 4% paraformaldehyde and imaged on n UltraVIEW VoX spinning disk confocal microscope (Nikon, Surrey, UK).

TABLE S1
Clinical isolates and strains

| Strain name | Genotype | Reference |
|---|---|---|
| ATCC 19606 | Clinical isolate | ATCC stock |
| hyr1Δ | hyr1Δ::hisG/ | Bailey et al. 1996 |
| | hyr1Δ:hisG-URA-3-hisG | |
| hyr1Δ + HYR1 | hyr1::hisG/ | Belmonte |
| | hyr1::hisG/RPS1/ | (unpublished) |
| | rps1::HYR1 | |
| C. albicans | Clinical isolate | Gillum et al. 1984 |
| SC5314 | | |
| C. glabrata | Clinical isolate | Odds et al. 2007 |
| SCS71182B | | |
| C. tropicalis | Clinical isolate | Clinical isolate from |
| AM2005/0546 | | Aberdeen |
| C. lusitaniae | Clinical isolate | Odds et al. 2007 |
| SCS211362H | | |
| C. krusei | Clinical isolate | Odds et al. 2007 |
| SCS71987M | | |
| C. parapsilosis | Clinical isolate | Rudek 1978 |
| ATCC22019 | | |
| C. dubliniensis | Clinical isolate | Moran et al. 1998 |
| CD36 | | |
| P. aeruginosa | Clinical isolate | ATCC stock |
| ATCC27853 | | |
| E. coli | Clinical isolate | Clinical isolate from |
| AM2002/0068 | | Aberdeen |
| S. marcescens | Clinical isolate | Flyg et al. 1980 |
| Db10 | | |

TABLE S2
Recombinant Hyr1 protein amino acid sequence.
The leader sequence is underlined and the 6xHis
tag is in italics, and is followed by the linker
‘G’. Hyr1 protein amino acids 63-350 make up
the remainder of the sequence.

| Recombinant | |
|---|---|
| protein | Amino acid sequence |
| antigen name | (amino acids 63-350) |
| Recombinant Hyr1 | METDTLLLWVLLLWVPGSTGGSGHHHHHHG |
| N-terminus | EVEKGASLFIKSDNGPVLALNVALSTLVRP |
| fragment | VINNGVISLNSKSSTSFSNFDIGGSSFTNN |
| | GEIYLASSGLVKSTAYLYAREWTNNGLIVA |
| | YQNQKAAGNIAFGTAYQTITNNGQICLRHQ |
| | DFVPATKIKGTGCVTADEDTWIKLGNTILS |
| | VEPTHNFYLKDSKSSLIVHAVSSNQTFTVH |
| | GFGNGNKLGLTLPLTGNRDHFRFEYYPDTG |
| | ILQLRAAALPQYFKIGKGYDSKLFRIVNSR |
| | GLKNAVTYDGPVPNNEIPAVCLIPCTNGPS |
| | APESESDLNTPTTSSIGT |

TABLE S3
Purified recombinant human IgG1 mAbs generated
using the single B cell technology.

| Antibody | Yield (mg) | Target |
|---|---|---|
| AB-120 | 12 | Hyr1 protein |
| AB-121 | 28.5 | Hyr1 protein |
| AB-122 | 67.9 | Hyr1 protein |
| AB-123 | 67.3 | Hyr1 protein |

TABLE VH

| | | | | | | | | | SEQ |
|---|---|---|---|---|---|---|---|---|---|
| AB | | | VH | | | | | | ID |
| name | | VH FW1 | CDR1 | VH FW2 | VH CDR2 | VH FW3 | VH CDR3 | VH FW4 | NO |
| 06-AB- | VH1 | EVQLVQSGGGLVQPG | TSYA | WVRQAPGK | VITGNVGTSY | RFTISRDNSKKTVSLQMNS | TRYDFSSGYY | WGQGTLVS | 15C |
| 120 | | GSLGLSCAASGFIF | MT | GLEWVS | YADSVKG | LRAEDTAIYYCVK | FDD | VSS | |
| 06-AB- | VH3 | EVQLVESGGTLVQPG | SDY | WVRQAPGK | NIKQDGSEKY | RVTISRDNAQNSVFLQMHS | DGYTFGPATT | WGRGTLVS | 15D |
| 121 | | GSLRLSCAASGFTF | WMN | GLEWVA | YVDSLRG | LSVEDTAVYYCAR | ELDH | VSS | |
| 06-AB- | VH3 | EVQLVQSGGGLAQPG | DDFA | WVRQPPGK | GLTWNGGSID | RFTISRDNAKNSLFLQMNS | GLSGGTMAPF | WGQGTMVS | 15E |
| 122 | | RSLRLSCAASGFGF | MH | GLEWVS | YAGSVRG | LRAEDTALYYCAK | DI | VSS | |
| 06-AB- | VH3 | EVQLLESGGGVVQPG | SNYG | WVRQAPGK | VVWFDGSYK | RFTISRDNSKSTLYLQMNS | PIMTSAFDI | WGPGTMVS | 15F |
| 123 | | RSLRLSCAASGFTF | MH | GLEWVA | YYTDSVKG | LRAEDTAVYYCVS | | VSS | |

TABLE VL

| | | | | | | | | | SEQ |
|---|---|---|---|---|---|---|---|---|---|
| AB | | | | | VL | | | | ID |
| name | | VL FW1 | VL CDR1 | VL FW2 | CDR2 | VL FW3 | VL CDR3 | VL FW4 | NO |
| 06-AB- | VK1 | DIVMTQSPSSVS | RASQGIS | WYQQKPGE | AASSL | GVPSRFSGSGSGTDFTLTISS | QQANSFPIT | FGQGTRLQI | 16C |
| 120 | | ASVGDKVTITC | RWLA | APELLIY | QS | LQPEDFATYYC | | K | |
| 06-AB- | VL3 | QLVLTQPPSVSV | SGDELRN | WYQQKSGQ | QDNN | GIPERFSGSQSGDTATLTISG | QAWVSQTLV | FGGGTKLTV | 16D |
| 121 | | SPGQTASITC | KYTS | SPVLVIY | RPS | TQAVDEADYYC | | L | |
| 06-AB- | VL3 | QAGLTQPPSVSV | GGNNIGS | WYQQKPGQ | DDSD | GVPERFSGSNSGNTATLTISS | QVWDRSSDH | FGGGTRLTV | 16E |
| 122 | | APGQTATIPC | KHVH | APVAVVY | RPS | VEAGDEADYYC | FWL | L | |
| 06-AB- | VL2 | QLVLTQPPSASG | TGTSSDV | WYQHHPGK | EVSQR | GVPDRFSGSKSGNTASLTVS | SSYAGSVVL | FGGGTKLTV | 16F |
| 123 | | SPGQSVTISC | GGSNFVS | APKLMIY | PS | GLQADDEADYYC | | L | |

Antibody Sequences and Seq ID No.s

TABLE C
Antibody AB120

| | | Seq. |
|---|---|---|
| 06-AB-120 | Sequence | ID No. |
| VH FW1 | EVQLVQSGGGLVQPGGSLGLSCAASGFIF | 1C |
| VH CDR1 | TSYAMT | 2C |
| VH FW2 | WVRQAPGKGLEWVS | 3C |
| VH CDR2 | VITGNVGTSYYADSVKG | 4C |
| VH FW3 | RFTISRDNSKKTVSLQMNSLRAEDTAIYYCVK | 5C |
| VH CDR3 | TRYDFSSGYYFDD | 6C |
| VH FW4 | WGQGTLVSVSS | 7C |
| VL FW1 | DIVMTQSPSSVSASVGDKVTITC | 8C |
| VL CDR1 | RASQGISRWLA | 9C |
| VL FW2 | WYQQKPGEAPELLIY | 10C |
| VL CDR2 | AASSLQS | 11C |
| VL FW3 | GVPSRFSGSGSGTDFTLTISSLQPEDFATYYC | 12C |
| VL CDR3 | QQANSFPIT | 13C |
| VL FW4 | FGQGTRLQIK | 14C |

Table D
Antibody AB121

| | | Seq. |
|---|---|---|
| 06-AB-121 | Sequence | ID No. |
| VH FW1 | EVQLVESGGTLVQPGGSLRLSCAASGFTF | 1D |
| VH CDR1 | SDYWMN | 2D |
| VH FW2 | WVRQAPGKGLEWVA | 3D |
| VH CDR2 | NIKQDGSEKYYVDSLRG | 4D |
| VH FW3 | RVTISRDNAQNSVFLQMESLSVEDTAVYYCAR | 5D |
| VH CDR3 | DGYTFGPATTELDH | 6D |
| VH FW4 | WGRGTLVSVSS | 7D |
| VL FW1 | QLVLTQPPSVSVSPGQTASITC | 8D |
| VL CDR1 | SGDELRNKYTS | 9D |
| VL FW2 | WYQQKSGQSPVLVIY | 10D |
| VL CDR2 | QDNNRPS | 11D |
| VL FW3 | GIPERFSGSQSGDTATLTISGTQAVDEADYYC | 12D |
| VL CDR3 | QAWVSQTLV | 13D |
| VL FW4 | FGGGTKLTVL | 14D |

TABLE E
Antibody AB122

| | | Seq. |
|---|---|---|
| 06-AB-122 | Sequence | ID No. |
| VH FW1 | EVQLVQSGGGLAQPGRSLRLSCAASGFGF | 1E |
| VH CDR1 | DDFAMH | 2E |
| VH FW2 | WVRQPPGKGLEWVS | 3E |
| VH CDR2 | GLTWNGGSIDYAGSVRG | 4E |
| VH FW3 | RFTISRDNAKNSLFLQMNSLRAEDTALYYCAK | 5E |
| VH CDR3 | GLSGGTMAPFDI | 6E |
| VH FW4 | WGQGTMVSVSS | 7E |
| VL FW1 | QAGLTQPPSVSVAPGQTATIPC | 8E |
| VL CDR1 | GGNNIGSKHVH | 9E |
| VL FW2 | WYQQKPGQAPVAVVY | 10E |
| VL CDR2 | DDSDRPS | 11E |
| VL FW3 | GVPERFSGSNSGNTATLTISSVEAGDEADYYC | 12E |
| VL CDR3 | QVWDRSSDHFWL | 13E |
| VL FW4 | FGGGTRLTVL | 14E |

TABLE F
Antibody AB123

| | | Seq. |
|---|---|---|
| 06-AB-123 | Sequence | ID No. |
| VH FW1 | EVQLLESGGGVVQPGRSLRLSCAASGFTF | 1F |
| VH CDR1 | SNYGMH | 2F |
| VH FW2 | WVRQAPGKGLEWVA | 3F |
| VH CDR2 | VVWFDGSYKYYTDSVKG | 4F |
| VH FW3 | RFTISRDNSKSTLYLQMNSLRAEDTAVYYCVS | 5F |
| VH CDR3 | PIMTSAFDI | 6F |
| VH FW4 | WGPGTMVSVSS | 7F |
| VL FW1 | QLVLTQPPSASGSPGQSVTISC | 8F |
| VL CDR1 | TGTSSDVGGSNFVS | 9F |
| VL FW2 | WYQHHPGKAPKLMIY | 10F |
| VL CDR2 | EVSQRPS | 11F |
| VL FW3 | GVPDRFSGSKSGNTASLTVSGLQADDEADYYC | 12F |
| VL CDR3 | SSYAGSVVL | 13F |
| VL FW4 | FGGGTKLTVL | 14F |

### REFERENCES

9. Rader C. Chemically programmed antibodies. Trends. Biotechnol. 32, 186-97 (2014).

10. Yoon S, Kim Y-, Shim H, Chung J. Current perspectives on therapeutic antibodies. Biotechnol. Bioprocess Eng. 15, 709-15 (2010).

11. Carter P J. Introduction to current and future protein therapeutics: a protein engineering perspective. Exp. Cell Res. 317, 1261-1269 (2011).

12. Berry J D, Gaudet RG. Antibodies in infectious diseases: polyclonals, monoclonals and niche biotechnology. New Biotech. 28, 489-501 (2011).

13. Saylor C, Dadachova E, Casadevall A. Monoclonal antibody-based therapies for microbial diseases. Vaccine. 27, G38-46 (2009).

14. Wilson P C, Andrews S F. Tools to therapeutically harness the human antibody response. Nat. Rev. Immunol. 12, 709-19 (2012).

34. Carter P J. Potent antibody therapeutics by design. Nat. Rev. Immunol. 6, 343-357 (2006).

35. Tiller T. Single B cell antibody technologies. N. Biotechnol. 28, 453-457 (2011).

36. Smith K, Garman L, Wrammert J, Zheng N Y, Capra J D, Ahmed R, et al. Rapid generation of fully human monoclonal antibodies specific to a vaccinating antigen. Nat. Protoc. 4, 372-384 (2009).

37. Liao H X, Levesque M C, Nagel A, Dixon A, Zhang R, Walter E, et al. High-throughput isolation of immunoglobulin genes from single human B cells and expression as monoclonal antibodies. J. Virol. Methods. 158, 171-179 (2009).

38. Huang J, Doria-Rose N A, Longo N S, Laub L, Lin C-, Turk E, et al. Isolation of human monoclonal antibodies from peripheral blood B cells. Nat. Protoc. 8, 1907-1915 (2013).

39. Jiang X, Suzuki H, Hanai Y, Wada F, Hitomi K, Yamane T, et al. A novel strategy for generation of monoclonal antibodies from single B cells using RT-PCR technique and in vitro expression. Biotechnol. Prog. 22, 979-88 (2006).

40. Bailey D A, Feldmann P J F, Bovey M, Gow N A R, Brown A J P. The Candida albicans HYR 1 gene, which is activated in response to hyphal development, belongs to a gene family encoding yeast cell wall proteins. J. Bacteriol. 178, 5353-5360 (1996).

42. Lewis L E, Bain J M, Lowes C, Gillespie C, Rudkin F M, Gow N A R, et al. Stage specific assessment of Candida albicans phagocytosis by macrophages identifies cell wall composition and morphogenesis as key determinants. PLoS Pathog. 8, e1002578 (2012).

43. Rudkin F M, Bain J M, Walls C, Lewis L E, Gow N A R, Erwig L P. Altered dynamics of Candida albicans phagocytosis by macrophages and PMNs when both phagocyte subsets are present. mBio. 4, e00810-13. doi: 10.1128/mBio.00810-13 (2013).

53. Durocher Y, Perret S, Kamen A. High-level and high-throughput recombinant protein production by transient transfection of suspension-growing human 293-EBNA1 cells. Nucleic Acids Res. 30, e9 (2002).

54. McKenzie C G J, Koser U, Lewis L E, Bain J M, Mora-Montes H M, Barker R N, et al. Contribution of Candida albicans cell wall components to recognition by and escape from murine macrophages. Infect. Immun. 78, 1650-1658 (2010). Additional references for Table 51:

Brand A, MacCallum D M, Brown A J, Gow N A, Odds F C. Ectopic expression of URA3 can influence the virulence phenotypes and proteome of Candida albicans but can be overcome by targeted reintegration of URA3 at the RPS10 locus. Eukaryot Cell 3: 900-909. doi: 10.1128/ec.3.4.900-909.2004 (2004).

Bailey D A, Feldmann P J F, Bovey M, Gow N A R, Brown A J P. The Candida albicans HYR1 gene, which is activated in response to hyphal development, belongs to a gene family encoding yeast cell wall proteins. J. Bacteriol. 178, 5353-5360 (1996).

Gillum A M, Tsay E Y, Kirsch D R. Isolation of the Candida albicans gene for orotidine-5′-phosphate decarboxylase by complementation of S. cerevisiae ura3 and E. coli pyrF mutations. Mol Gen Genet. 198:179-8 (1984).

Odds F C, Hanson M F, Davidson A D, Jacobsen M D, Wright P, Whyte J A, Gow N A, Jones B L. One year prospective survey of Candida bloodstream infections in Scotland. J Med Microbiol. 56: 1066-1075 (2007).

Rudek W. Esterase activity in Candida species. J. Clin. Microbiol. 8: 756-759 (1978).

Moran, G. P., Sanglard D., Donnelly S. M., Shanley D. B., Sullivan D. J., Coleman D. C. Identification and expression of multidrug transporters responsible for fluconazole resistance in Candida dubliniensis. Antimicrob. Agents Chemother. 42, 1819-1830 (1998).

Flyg, C. et al. Insect Pathogenic Properties of Serratia marcescens: Phage-resistant Mutants with a Decreased Resistance to Cecropia Immunity and a Decreased Virulence to Drosophila. J Gen. Microb. 120(1): 173-181 (1980)

1. An isolated recombinant human anti-Candida antibody molecule derived from single B cells for use in a method of treatment of an Acinetobacter bacterial infection, wherein the antibody molecule comprises a VH domain comprising a HCDR3 having the amino acid sequence of SEQ ID NO: 6x or the sequence of SEQ ID NO: 6x with 1, 2, or 3 amino acid substitutions, deletions or insertions, wherein ‘x’ is one letter from F, E, D and C, and said sequence is as shown in Table ‘x’ herein.

2. An antibody molecule for use according to claim 1 wherein the antibody molecule comprises an HCDR2 having the amino acid sequence of SEQ ID NO: 4x or the sequence of SEQ ID NO: 4x with 1, 2, or 3 amino acid substitutions, deletions or insertions.

3. An antibody molecule for use according to claim 1 or claim 2 wherein the antibody molecule comprises an HCDR1 having the amino acid sequence of SEQ ID NO: 2x or the sequence of SEQ ID NO: 2x with 1, 2 or 3 amino acid substitutions, deletions or insertions.

4. An antibody molecule for use according to any one of claims 1-3 wherein the antibody molecule comprises a VH domain comprising a HCDR1, a HCDR2 and a HCDR3 having the sequences of SEQ ID NOs 2x, 4x and 6x respectively.

5. An antibody molecule for use according to any one of claims 1-4 wherein the antibody molecule comprises a VH domain comprising one or more or all of a FW1, a FW2, a FW3 and a FW4 having the sequences of SEQ ID NOs lx, 3x, 5x and 7x respectively.

6. An antibody molecule for use according to any one of claims 1-5 wherein the antibody molecule comprises a VH domain having an amino acid sequence at least about 80% identical to SEQ ID NO: 15x and\or having the amino acid sequence of SEQ ID NO: 15x and\or the sequence of SEQ ID NO: 15x with 1, 2, 3, 4, 5, 6, 7, 8, 9, or 10 amino acid substitutions, deletions or insertions in SEQ ID NO: 15x.

7. An antibody molecule for use according to any one of claims 1-6 wherein the antibody molecule comprises a VL domain comprising LCDR1, LCDR2 and LCDR3 having the sequences of SEQ ID NOs 9x, 11x and 13x respectively, or the sequences of SEQ ID NOs 9x, 11x and 13x respectively with, independently, 1, 2 or 3 or more amino acid substitutions, deletions or insertions.

8. An antibody molecule for use according to claim 7 wherein the antibody molecule comprises a VL domain comprising LCDR1, LCDR2 and LCDR3 having the sequences of SEQ ID NOs 9x, 11x and 13x respectively.

9. An antibody molecule for use according to any one of claims 1-8 wherein the antibody molecule comprises a VL domain comprising one or more or all of a FW1, a FW2, a FW3 and a FW4 having the sequences of SEQ ID NOs 8x, 10x, 12x and 14x respectively.

10. An antibody molecule for use according to any one of claims 1-9 wherein the antibody molecule comprises a VL domain having an amino acid sequence at least about 80% identical to SEQ ID NO: 16x and\or having the sequence of SEQ ID NO: 16x and\or the sequence of SEQ ID NO: 16x with 1 or more, for example 2, 3, 4, 5, 6, 7, 8, 9, 10 or more amino acid substitutions, deletions or insertions in SEQ ID NO: 16x.

11. An antibody molecule for use according to any one of claims 1-10 wherein the antibody molecule comprises a VH domain comprising a HCDR1, a HCDR2 and a HCDR3 having the sequences of SEQ ID NOs 2x, 4x, and 6x, respectively, and a VL domain comprising a LCDR1, a LCDR2 and a LCDR3 having the sequences of SEQ ID NOs 9x, 11x and 13x, respectively.

12. An antibody molecule for use according to any one of claims 1-11 wherein the antibody molecule comprises VH and VL domains having the amino acid sequences of SEQ ID NO: 15x and SEQ ID NO: 16x respectively.

13. An antibody molecule for use according to any one of claims 1-12, wherein the bacterial infection is an A. baumannii infection.

14. An antibody molecule for use according to any one of claims 1-12, wherein the bacterial infection is an A. Baumannii infection, an A. pittii infection, an A. nosocomialis infection, an A. calcoaceticus infection, an A. seifertii infection or an A. dijkshoorniae infection.

15. An antibody molecule for use according to anyone of claims 1-13, wherein the antibody molecule binds A. baumannii with an EC₅₀ value of (a) 1 to 1500 ng/mL; or (b) less than 20 ng/mL.

16. An antibody molecule for use according to any one of claims 1-15 wherein the antibody molecule is a whole antibody or a scAb.

17. An antibody molecule for use according to any one of claims 1-16 wherein the antibody molecule comprises a payload which is cytotoxic.

18. A pharmaceutical composition for use in a method of treatment of an Acinetobacter bacterial infection, the composition comprising an antibody molecule as defined in any one of claims 1-17 and a pharmaceutically acceptable excipient.

19. A composition of matter for use in in a method of treatment of an Acinetobacter bacterial infection, the composition comprising (1) a pharmaceutical composition as defined in claims 18 and (2) a further antibacterial agent.

20. A method of identifying or labelling an Acinetobacter cell, the method comprising contacting the cell with an antibody molecule as defined in any one of claims 1-16.

21. A method of opsonising, or increasing the rate of opsonisation of, an Acinetobacter cell, the method comprising contacting or pre-incubating the cell with an antibody molecule as defined in any one of claims 1-16.

22. A method of increasing the rate of engulfment of an Acinetobacter cell, the method comprising contacting the cell with an antibody molecule as defined in any one of claims 1-16.

23. A method of treatment of an Acinetobacter bacterial infection, comprising administering an antibody molecule as defined in any one of claims 1-17, or a composition as defined in claim 18 or claim 19, to an individual in need thereof.

24. The method of claim 23, wherein the Acinetobacter bacterial infection is an A. baumannii bacterial infection.

25. The method of claim 23, wherein the bacterial infection is an A. Baumannii infection, an A. pittii infection, an A. nosocomialis infection, an A. calcoaceticus infection, an A. seifertii infection or an A. dijkshoomiae infection.

26. The method of claims 23-25, wherein the treatment comprises administering a second antibacterial agent, wherein the second antibacterial agent is optionally:
(a) a cephalosporin;
(b) a combination beta-lactam/beta-lactamase inhibitor (optionally sulbactam);
(c) a carbapenem (optionally meropenem, doripenem, or imipenem);
(d) a polymyxin (optionally colistin or polymixin B);
(e) tigecycline; or
(f) minocycline.

27. Use of an antibody molecule as defined in any one of claims 1-17 in the manufacture of a medicament for use in treating or preventing an Acinetobacter infection.

28. An antibody molecule for use according to any one of claims 1-17, a composition for use according to any one of claim 18 or claim 19, a method according to any one of claims 23-26, or a use according to claim 27, wherein the Acinetobacter bacterial infection is in an immunosuppressed individual.

29. A method for detecting the presence or absence of a bacterium which is Acinetobacter spp, the method comprising
(i) contacting a sample suspected of containing the bacterium with an antibody molecule as defined in any one of claims 1-16, and
(ii) determining whether the antibody molecule binds to the sample, wherein binding of the antibody molecule to the sample indicates the presence of the bacterium.

30. A method for diagnosing a bacterial infection in an individual which is caused by Acinetobacter spp, the method comprising
(i) contacting a biological sample obtained from the individual with an antibody molecule as defined in any one of claims 1-16, and
(ii) determining whether the antibody molecule binds to the biological sample, wherein binding of the antibody molecule to the biological sample indicates the presence of a bacterial infection.

31. A linear flow device (LFD) for detecting an analyte which is a bacterial pathogen in a sample fluid,
wherein said LFD comprises:
(i) a housing, and
(ii) at least one flow path leading from a sample well to a viewing window, wherein said flow path comprises one or more carriers along which the sample fluid is capable of flowing by capillary action, the or each carrier comprising an analyte-detecting means;
wherein the presence of analyte produces a line in the viewing window which indicates an analyte concentration,
wherein the bacterial pathogen is Acinetobacter spp. and the at least one analyte-detecting means is an antibody molecule as defined in any one of claims 1-16.

32. A device as claimed in claim 31 which further comprises a control zone capable of indicating the assay has been successfully run.

33. A device as claimed in claim 31 or claim 32 having a plurality of analyte-detecting means capable of distinguishing between multiple bacterial pathogens, wherein one of the analyte-detecting means is an antibody molecule as defined in any one of claims 1-16.

34. A device as claimed in claim 33 wherein the multiple bacterial pathogens comprise A. baumannii, plus one or more or all of Pseudomonas aeruginosa, Escherichia coli and Serratia marcescens.

35. A device as claimed in claim 33 wherein the multiple bacterial pathogens comprise A. pittii, A. nosocomialis, A. calcoaceticus, A. seifertii or A. dijkshoomiae, plus one or more or all of Pseudomonas aeruginosa, Escherichia coli and Serratia marcescens.

36. The device of any one of claims 31-35, for use in a method of any one of claims 23-26.

37. Use of an antibody molecule as defined in any one of claims 1-16 for the prevention of biofilm formation between A. baumannii and C. albicans.
